(19) United States
(12) Patent Application Publication
(10) Pub. No.: US 2006/0083781 A1
(43) Pub. Date: Apr. 20, 2006

(54) Functionalized solid lipid nanoparticles and methods of making and using same

(76) Inventors: V. Prasad Shastri, Nashville, TN (US); Eric Sussman, Seattle, WA (US); Ashwath Jayagopal, Nashville, TN (US)

Correspondence Address: NEEDLE & ROSENBERG, P.C., SUITE 1000, 999 PEACHTREE STREET, ATLANTA, GA, 30309-3915, US

(21) Appl. No.: 11/251,109

(22) Filed: Oct. 14, 2005

Related U.S. Application Data
(60) Provisional application No. 60/618,962, filed on Oct. 14, 2004.
(60) Provisional application No. 60/658,520, filed on Mar. 3, 2005.
(60) Provisional application No. 60/722,132, filed on Sep. 30, 2005.

(51) Int. Cl. A61K 9/127

(52) U.S. Cl. 424/450; 977/907

(57) ABSTRACT

In one aspect, the invention relates to functionalized solid lipid nanoparticles comprising a neutral lipid and a first functionalized polymer comprising at least one ionic or ionizable moiety and methods for providing same. In a further aspect, the invention relates to tumor targeting therapeutic systems, multimodal diagnostic therapeutic systems, thermoresponsive payload delivery systems, magnetic-driven targeting systems, therapeutic diagnostic systems, stabilized ink compositions, and cosmetic formulations comprising the solid lipid nanoparticles of the invention. In a further aspect, the invention relates to methods of delivering at least one biologically active agent, pharmaceutically active agent, magnetically active agent, or imaging agent across the blood-brain barrier, across a cellular lipid bilayer and into a cell, and to a subcellualr structure. This abstract is intended as a scanning tool for purposes of searching in the particular art and is not intended to be limiting of the present invention.

### PRIORITY CLAIM

This application claims the benefit of U.S. application Ser. No. 60/618,962, filed Oct. 14, 2004; U.S. Application No. 60/658,520, filed Mar. 3, 2005; and U.S. Application No. 60/722,132, filed Sep. 30, 2005, which are all hereby incorporated herein by reference in their entireties.

### BACKGROUND

Solid lipid nanoparticles (SLN) were developed at the beginning of the 1990s as an alternative carrier system to emulsions, liposomes, and polymeric nanoparticles. SLN can provide advantages including stabilization of incorporated compounds, controlled release, occlusivity, and film formation on skin, including in vivo effects on the skin. SLN are conventionally prepared by a melting/solidification process, wherein the lipid is first melted, dispersed in water and then cooled to solidify the lipid particles. Alternatively, SLN are conventionally produced using an emulsion process akin to the formation of polymeric microparticles, wherein the lipids are dissolved in a solvent, emulsified, and then dispersed in an aqueous solution containing an emulsifying agent to harden the solid lipid nanoparticles. The role of the emulsifying agent is to stabilize the SLN; however, it also precludes further functionalization of the SLN. SLN are generally known to those of skill in the art and may be obtained by conventional methods as described in, for example, M. R. Gasco, Nanoparticelle Lipidiche Solide Quali Sistemi Terapeutici Colloidali, NCF nr. 7, 1996, pg 71-73; Kozariara et al., In-situ Blood-Brain Barrier Transport of Nanoparticles, Pharmaceutical Research, vol. 20, no. 11, p. 1772 (2003); and Lockman, et al., Brain Uptake of Thiamine-Coated Nanoparticles, Journal of Controlled Release, 93 (2003) 271-282.

However, SLN prepared by conventional means generally require the use of surfactants or emulsifiers, typically fail to achieve stable aqueous suspensions, and/or fail to provide satisfactory surface functionalization. Therefore, there remains a need for methods and compositions that overcome these deficiencies and that effectively provide functionalized solid lipid nanoparticles.

### SUMMARY

In accordance with the purpose(s) of the invention, as embodied and broadly described herein, the invention, in one aspect, relates to solid lipid nanoparticles comprising a neutral lipid and a first functionalized polymer, wherein the solid lipid nanoparticle has a surface, an interior, an exterior, and a diameter; wherein the first functionalized polymer comprises a polymer having at least one ionic or ionizable pendant group, a polymer having at least one ionic moiety in the polymer backbone, or a copolymer thereof, or mixture thereof; wherein at least a portion of the first functionalized polymer is at the exterior of the solid lipid nanoparticle; and wherein the diameter of the solid lipid nanoparticle is from about 10 nm to about 1,000 nm.

In a further aspect, the invention relates to a solid lipid nanoparticle comprising a neutral lipid and a polyether; wherein the solid lipid nanoparticle has an interior, an exterior, and a diameter; wherein at least a portion of the polyether is at the exterior of the solid lipid nanoparticle; and wherein the diameter of the solid lipid nanoparticle is from about 10 nm to about 1,000 nm. In a further aspect, the solid lipid nanoparticle can further comprise at least one of a biologically active agent, a pharmaceutically active agent, a magnetically active agent, or an imaging agent, or a mixture thereof.

In a further aspect, the invention relates to a functionalized quantum dot comprising one or more quantum dots encapsulated within the solid lipid nanoparticle of the invention. In a further aspect, the first functionalized layer of the functionalized quantum dot can further comprise at least one cysteine-rich protein, at least one metallothionein-rich protein, or a mixture thereof.

In a further aspect, the invention relates to tumor targeting therapeutic systems comprising the solid lipid nanoparticle of the invention and a pharmaceutically active agent encapsulated within the solid lipid nanoparticles; wherein the biologically active agent comprises at least one enzyme.

In a further aspect, the invention relates to multimodal diagnostic therapeutic systems comprising at least one solid lipid nanoparticle of the invention.

In a further aspect, the invention relates to multimodal diagnostic therapeutic systems comprising a liposome comprising at least one solid lipid nanoparticle of the invention encapsulated within the liposome, optionally further comprising a biologically active agent, a pharmaceutically active agent, a magnetically active agent, imaging agent, or a mixture thereof encapsulated within the liposome.

In a further aspect, the invention relates to multimodal diagnostic therapeutic systems comprising a microsphere comprising at least one solid lipid nanoparticle of the invention encapsulated within the microsphere, optionally further comprising a delivery package, such as a biologically active agent, a pharmaceutically active agent, a magnetically active agent, imaging agent, or a mixture thereof encapsulated within the microsphere.

In a further aspect, the invention relates to thermoresponsive payload delivery systems comprising a first solid lipid nanoparticle, wherein the first solid lipid nanoparticle has a first payload and a first melting temperature, optionally further comprising a second solid lipid nanoparticle, wherein the second solid lipid nanoparticle has a second payload and a second melting temperature, and wherein the second melting temperature is higher than the first melting temperature.

In a further aspect, the invention relates to methods of thermoresponsive payload delivery within a subject comprising the steps of administering an effective amount of the thermoresponsive drug delivery systems of the invention to a subject; applying heat to a location within the subject, thereby increasing the temperature of the location above the first melting temperature and melting the solid lipid nanoparticle of the invention, whereby the first payload is delivered to the location within the subject.

In a further aspect, the invention relates to methods of thermoresponsive payload delivery within a subject comprising the steps of administering an effective amount of the thermoresponsive drug delivery systems of the invention to a subject; applying a first heat to a first location within the subject, thereby increasing the temperature of the first location above the first melting temperature and melting the first solid lipid nanoparticle, whereby the first payload is delivered to the first location within the subject; and applying a second heat to a second location within the subject, thereby increasing the temperature of the second location above the second melting temperature and melting the second solid lipid nanoparticle, whereby the second payload is delivered to the second location within the subject.

In a further aspect, the invention relates to methods of providing the solid lipid nanoparticles of the invention comprising the steps of providing an organic phase comprising: (1) a binary solvent system and (2) a neutral lipid; providing an aqueous phase comprising water and at least one first functionalized polymer having at least one ionic or ionizable moiety; and combining the organic phase and the aqueous phase, optionally further comprising one or more of the steps of separating the organic phase from the aqueous phase, separating at least a portion of the organic phase from the aqueous phase, and/or admixing with the product a second functionalized polymer having at least one ionic or ionizable moiety that is complementary to the ionic or ionizable moiety of the first functionalized polymer.

In a further aspect, the invention relates to the products produced by the methods of the invention.

In a further aspect, the invention relates to methods of delivering at least one biologically active agent, pharmaceutically active agent, magnetically active agent, or imaging agent across the blood-brain barrier comprising the step of administering an effective amount of the solid lipid nanoparticle of the invention to a subject, whereby the at least one biologically active agent, pharmaceutically active agent, magnetically active agent, or imaging agent is delivered across the blood brain barrier.

In a further aspect, the invention relates to methods of delivering at least one biologically active agent, pharmaceutically active agent, magnetically active agent, or imaging agent to a location within a subject comprising the steps of administering an effective amount of the solid lipid nanoparticle of the invention to a subject, applying a magnetic field to the location, whereby the at least one biologically active agent, pharmaceutically active agent, magnetically active agent, or imaging agent is delivered to the location.

In a further aspect, the invention relates to methods of delivering at least one biologically active agent, pharmaceutically active agent, magnetically active agent, or imaging agent across a cellular lipid bilayer and into a cell comprising the step of introducing the solid lipid nanoparticle of the invention proximate to the exterior of the cell, whereby the at least one biologically active or pharmaceutically active agents is delivered across the cellular lipid bilayer and into the cell.

In a further aspect, the invention relates to methods of delivering at least one pharmaceutically active agent, magnetically active agent, or imaging agent to a subcellular organelle comprising the step of introducing the solid lipid nanoparticle of the invention proximate to the exterior of the cell, wherein the solid lipid nanoparticle further comprises at least one pharmaceutically active agent, magnetically active agent, or imaging agent, and wherein the biologically active agent comprises a signal protein specific for the organelle, whereby the at least one pharmaceutically active agents is delivered across the cellular lipid bilayer and into the cell.

In a further aspect, the invention relates to therapeutic diagnostic systems comprising a hydrophobic polymer substrate and the solid lipid nanoparticles of the invention adsorbed on the surface of the substrate.

In a further aspect, the invention relates to methods of providing the therapeutic diagnostic system comprising the step of contacting an aqueous suspension of the solid lipid nanoparticle of the invention with a hydrophobic polymer substrate.

In a further aspect, the invention relates to methods of modulating particle size of the solid lipid nanoparticles of the invention comprising the steps of selecting a binary solvent system; dissolving a neutral lipid in the binary solvent system, thereby producing an organic phase; providing an aqueous phase comprising a first functionalized polymer; and combining the organic phase and the aqueous phase, thereby producing a substantially monodisperse solid lipid nanoparticle suspension.

In a further aspect, the invention relates to the solid lipid nanoparticles of the invention, further comprising a dye, a pigment, or a colorant, and stabilized ink compositions comprising the solid lipid nanoparticles of the invention and a dye, a pigment, or a colorant.

In a further aspect, the invention relates to cosmetic formulations comprising the solid lipid nanoparticles of the invention and an active ingredient having cosmetic activity, pharmaceutical activity, or both.

In a further aspect, the invention relates to methods for the treatment of the upper layers of the epidermis comprising the step of topically administering to a subject an amount effective to treat the upper layers of the epidermis of a composition comprising the cosmetic formulations of the invention.

In a further aspect, the invention relates to a system for delivery of a pharmaceutically active agent across the blood brain barrier, comprising (1) a solid lipid nanoparticle, (2) a surface functional layer surrounding the nanoparticle, and (3) a pharmaceutically active agent, whereby the pharmaceutically active agent is capable of being delivered across the blood brain barrier. In a further aspect, the surface functional layer comprises poly(acrylic acid), poly-L-lysine, polyglycine, polyethylene glycol, heparin, hydroxypropylmethylcellulose, hydroxyethylcellulose, hydroxypropylcellulose, polyvinylpyrrolidone, polyvinyl alcohol, a methacrylic acid copolymer, an ethyl acrylate-methyl methacrylate copolymer, or a mixture thereof.

In a further aspect, the invention relates to a method of delivering a pharmaceutically active agent across the blood brain barrier, comprising (1) providing a system comprising a solid lipid nanoparticle, a surface functional layer a surrounding the nanoparticle, and a pharmaceutically active agent; and (2) administering the nanoparticle to a subject, whereby the pharmaceutically active agent is delivered across the blood brain barrier.

In a further aspect, the invention relates to a lipid-encapsulated quantum dot comprising (1) a solid lipid nanoparticle, (2) a coating bearing functionality at the surface of the solid lipid nanoparticle, and (3) a quantum dot, wherein the lipid-encapsulated quantum dot has a surface. In a further aspect, the coating comprises poly(styrene sulfonate), poly-L-lysine, polyethylene glycol, or heparin. In a further aspect, the coating comprises a metallothionein or comprises cysteine-rich peptide segments. In one aspect, the coating bearing functionality at the surface of the solid lipid nanoparticle can be, for example, a first functionalized polymer. In a further aspect, the coating bearing functionality at the surface of the solid lipid nanoparticle can further comprise a second functionalized polymer. In a further aspect, the coating bearing functionality at the surface of the solid lipid nanoparticle can further comprise a third functionalized polymer.

In a further aspect, the invention relates to a method of delivering a quantum dot into a cell comprising the step of administering the lipid-encapsulated quantum dot of the invention to a subject, whereby the quantum dot is delivered into the cell.

In a further aspect, the invention relates to a solid lipid nanoparticle comprising: a neutral lipid and a first functionalized polymer, wherein the nanoparticle has a surface, an interior, an exterior, and a diameter; wherein the first functionalized polymer comprises a polyether, a polymer having at least one ionic or ionizable pendant group, a polymer having at least one ionic moiety in the polymer backbone, or a copolymer thereof, or mixture thereof; wherein at least a portion of the first functionalized polymer is concentrated at the exterior of the nanoparticle; and wherein the diameter of the nanoparticle is from about 10 nm to about 1,000 nm.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying figures, which are incorporated in and constitute a part of this specification, illustrate several embodiments and together with the description serve to explain the principles of the invention.

FIG. 1 shows a graph illustrating the relationship between particle size and solvent polarity parameter for the solid lipid nanoparticles of the invention.

FIG. 2 shows lipid-encapsulated quantum dot nanoparticle diameter as measured by Beckman-Coulter Delsa 440SX zetasizer analysis. Large NMP/Acetone ratios in the organic phase result in smaller particles, likely due to the rapid influx of water that is promoted. Lower NMP volume fractions reduce the phase's miscibility with water, thus allowing for larger nanoparticle formation.

FIG. 3 shows the tunability of SLN size: SLN size can be tuned by varying the binary organic solvent composition. SPP—Solvent Polarity Parameter.

FIG. 4 shows SLN functionalized with a primary negatively charged layer, followed by a secondary layer of positively charged species (polycation), and followed by a tertiary layer of negatively charged moieties (polyanion).

FIG. 5 shows a comparison of two lipid encapsulated quantum dot constructs. Larger diameters (left panel) result in the entrapment of a greater number of quantum dots compared to smaller diameters (right panel), resulting in an increased emission intensities.

FIG. 6 shows fluorescence microscopy (100× total magnification) of confluent bovine aortic endothelial cell (BAEC) monolayer incubated with lipid-encapsulated quantum dots (580 nm) for 2 hours followed by rinsing 3 times in PBS.

FIG. 7 shows staining of live BAEC with lipid-encapsulated quantum dots analyzed by confocal laser scanning microscopy. Image indicates perinuclear staining of 580 nm emitting nanocrystals.

FIG. 8 shows confocal laser scanning microscopy (Zeiss LSM Meta 5) of BAEC incubated with lipid-encapsulated quantum dots and trypsinized indicates retention of nanocrystals. This indicates that the lipid probes are retained in the cytoplasm rather than the plasma membranes of the cells. Photo is taken as cells are reattaching to the surface (i.e., right after trypsinization).

FIG. 9 shows live fluorescence imaging of BAEC incubated with lipid-encapsulated quantum dots (580 nm). Left: At onset of lipid probe injection, quantum dot fluorescence is observed above the cell monolayer, with rapid Brownian motion observed in solution. Circles indicate quantum dots in rapid Brownian motion. Right: After 10 minutes of incubation, quantum dot Brownian motion nearly ceases, with most lipid probes already becoming embedded in cell plasma membranes and cytosol. This process was also observed at 4° C., indicating that the uptake of lipid probes is energy-independent.

FIG. 10 shows flow cytometry of BAEC incubated with either lipid-encapsulated quantum dots (580 nm, 100 nM) or 20 μL 100 mM PBS for 10 minutes at 37° C. Cells were trypsinized prior to flow cytometric analysis, which would remove all plasma membrane-bound material. Enhanced fluorescence was observed in incubated cells compared to control, unlabeled cells, indicating that lipid-encapsulated quantum dots can label a large number of viable cells. Cells analyzed on a BD FACSCalibur in FL2 with 488 nm excitation laser.

FIG. 11 shows flow cytometry of BAEC at 4C. Left: Unlabeled BAEC. Right: Incubated at 4C with 580 nm emitting lipid encapsulated quantum dots at 100 nm. Incubation was for 25 minutes. Analysis was performed on a BD LSR II flow cytometer with 405 nm excitation with a 585/42 nm bandpass filter.

FIG. 12 shows the relationship between emission wavelength and fluorescence intensity for the SLN-QD.

FIG. 13 shows zeta potential measurements for SLN-QD functionalized with PSS. The isoelectric point of the colloids corresponds to the pKa of the sulfonic acid group of PSS, indicating that PSS is present on the SLN-QD surface.

FIG. 14 shows a schematic of transport studies used in connection with the solid lipid nanoparticles of the invention.

FIG. 15 shows transport of albumin across bovine aortic endothelial cell monolayer.

FIG. 16 shows transport of coumarin across bovine aortic endothelial cell monolayer.

FIG. 17 shows a T1-contrast enhancement graph when encapsulated Gd-DTPA is injected systemically for imaging of mouse brain. This shows that signal was detected in the brain following tail vein injection of the SLN-Gd-DTPA particles.

FIG. 18 shows FITC-BSA/iron oxide nanoparticles entrapped within a lipid matrix, in 0.5M trypan blue solution. Trypan blue quenches FITC fluorescence, thus indicating that FITC-BSA is successfully encapsulated by the lipid and protecting from the aqueous environment.

FIG. 19 shows fluorescence microscopic analysis of CdSe/ZnS 580 nm peak emission quantum dot stability in aqueous environments. Quantum dot specimens were pipetted at 100 uL on a MatTek glass bottom dish and observed on a Nikon TE2000U using specific QD580 filter sets (Chroma Corp.). Concentrations were approximately 10 nM. Top left: Unencapsulated quantum dots in toluene; Top right: unencapsulated quantum dots in toluene at instant exposure to a 10 uL injection of lactated Ringers'. Bottom left: lipid-encapsulated quantum dots in water; Bottom right: lipid-encapsulated quantum dots exposed to Ringers' exhibit no aggregation and disintegration.

FIG. 20 shows Philips CM-12 Transmission electron microscopy of quantum dots encapsulated using Softisan 100 lipid. Specimens were stained with phosphotungstic acid to stain the lipid coating in negative relief. Quantum dots appear as electron dense species within the lipid matrix. Within the lipid, quantum dots are observed to be well-dispersed, with no aggregation into a quantum “ball” being visible. Left: 66000× at 80 keV; Right: 175000× at 80 keV.

FIG. 21 shows a graph showing zeta potential of the solid lipid nanoparticles of the invention as a function of pH.

FIG. 22 shows: Left: TEM analysis SLN-QD indicate entrapped, disperse QD within a lipid matrix as stained by PTA. Middle, Right: Fluorescence microscopy indicates that approximately uniformly sized SLN-QD are achieved by the process; adjustment of NMP:Acetone ratios can yield large (middle) or small (right) SLN-QD.

FIG. 23 shows fluorescence microscopic analysis of CdSe/ZnS 580 nm peak emission quantum dot stability in aqueous environments. Quantum dot specimens were pipetted at 100 uL on a MatTek glass bottom dish and observed on a Nikon TE2000U using specific QD580 filter sets (Chroma Corp.) Concentrations were approximately 10 nM. Top left: Unencapsulated quantum dots in toluene; Top right: unencapsulated quantum dots in toluene at instant exposure to a 10 uL injection of lactated Ringers'. Bottom left: lipid-encapsulated quantum dots in water; Bottom right: lipid-encapsulated quantum dots exposed to Ringers' exhibit no aggregation and disintegration.

FIG. 24 shows a micrograph of T lymphocytes internalized with lipid-coated QD of the invention attached to CPPs.

FIG. 25 shows another micrograph of T lymphocytes internalized with lipid-coated QD of the invention attached to CPPs.

FIG. 26 shows another micrograph of T lymphocytes internalized with lipid-coated QD of the invention attached to CPPs.

FIG. 27 shows another micrograph of T lymphocytes internalized with lipid-coated QD of the invention attached to CPPs.

### DETAILED DESCRIPTION

The present invention may be understood more readily by reference to the following detailed description of aspects of the invention and the Examples included therein and to the Figures and their previous and following description.

Before the present compounds, compositions, articles, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular embodiments only and is not intended to be limiting.

A. Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein may be different from the actual publication dates, which may need to be independently confirmed.

As used in the specification and the appended claims, the singular forms “a,” “an” and “the” include plural referents unless the context clearly dictates otherwise. Thus, for example, reference to “a component,” “a polymer,” or “a particle” includes mixtures of two or more such components, polymers, or particles, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

By the term “effective amount” of a compound or property as provided herein is meant such amount as is capable of performing the function of the compound or property for which an effective amount is expressed. As will be pointed out below, the exact amount required will vary from process to process, depending on recognized variables such as the compounds employed and the processing conditions observed. Thus, it is not possible to specify an exact “effective amount.” However, an appropriate effective amount may be determined by one of ordinary skill in the art using only routine experimentation.

As used herein, the terms “Optional” or “optionally” means that the subsequently described event or circumstance may or may not occur, and that the description includes instances where said event or circumstance occurs and instances where it does not.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. In particular, “administration” can be by bolus injection with a syringe and needle, or by infusion through a catheter in place within a vessel. A vessel can be an artery or a vein. Administration can be continuous or intermittent. In one aspect, systemic delivery of payloads by transdermal administration into subcutaneous circulation using the solid lipid nanoparticles of the invention can be accomplished in combination with a chemical penetration enhancer.

As used herein, the term “copolymer” means a polymer formed from two or more polymers. By way of example and without limitation, a copolymer can be an alternating copolymer, a random copolymer, a block copolymer, or a graft copolymer.

As used herein, the term “microsphere” means any microscale delivery system known to those of skill in the art. The term also includes microcapsules.

As used herein, the term “binary solvent” means a solvent system comprising two or more miscible or partially miscible solvents. This term specifically includes ternary, four solvent, and five solvent systems. Typically, the solvent systems comprises solvents that are liquids at room temperature and at atmospheric pressure; however, it is also understood that one or more of the solvents in the system can be a solid or gas at room temperature and at atmospheric pressure, while the overall system is a liquid at room temperature and at atmospheric pressure.

As used herein, the term “biologically active agent” or “bioactive agent” means an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, hormones, antibodies, or therapeutic proteins. Other bioactive agents include prodrugs, which are agents that are not biologically active when administered but, upon administration to a subject are converted to bioactive agents through metabolism or some other mechanism. Examples of biologically active agents that can be used in connection with the invention include, without limitation, one or more of biotin, streptavidin, protein A, protein G, an antibody, antibody fragment F(ab)2, antibody fragment F(ab)′, a receptor ligand such as VEGF, VLA-4, or TNF-alpha, a neurotransmitter such as serotonin, a receptor antagonist such as muscimol (GABA antagonist), or an antioxidants such as Vitamin E (alpha-tocopherols) or C (ascorbic acid). Additionally, any of the compositions of the invention can contain combinations of two or more bioactive agents.

As used herein, the term “pharmaceutically active agent” includes a “drug” or a “vaccine” and means a molecule, group of molecules, complex or substance administered to an organism for diagnostic, therapeutic, preventative medical, or veterinary purposes. This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA). Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a radiosensitizer, the combination of a radiosensitizer and a chemotherapeutic, a steroid, a xanthine, a beta-2-agonist bronchodilator, an antiinflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythtnic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic antiinflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-l-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

As used herein, the term “targeting protein” refers to an antibody targeted toward a specific antigen., for example, on tumor cell surfaces (tumor-associated antigens), endothelial cell surfaces (e.g., VCAM-1, PECAM-1, ICAM-1 IgG superfamily of proteins), and white and red blood cell surfaces. An antibody can be, for example, therapeutically inhibitory by competing with the actual ligand for the receptor binding slot (e.g., TNF-alpha is therapeutically inhibited by the commercially-available Remicade (infliximab) monoclonal antibody), or an antibody can be used to identify the presence/absence of a biomarker. For example, a biologically active antibody can be used to quantitatively estimate the number of tumor associated antigens on a particular tumor cell type or to count the number of endothelial surface proteins on a cell in response to inflammatory stimuli, in effect “scoring” the progression of a disease or the response of the body to therapeutic interventions. A targeting protein can also include an internalization peptide. For example, VCAM-1 receptors expressed on endothelial cell surfaces lining blood vessels can be targeted specifically by custom peptides bound to NPs, and upon binding, the nanoparticle-peptide conjugate can be subsequently internalized into the cell expressing the receptor. Similar applications have been investigated for cancer treatments. Furthermore, internalization peptide includes cell penetrating peptides (CPP), which are nonspecifically internalized into many cell types. These peptides can enhance SLN transport into cells and/or target subcellular organelles.

As used herein, the term “signal protein” refers to a protein that serves as a ligand to a receptor. This function can be used to promote a certain biological activity. For example, by surface presentation of TNF-alpha to the nanoparticle surface, inflammation/immune response can in effect be activated in immuno-compromised individuals. In a further aspect, VEGF can be presented on a nanoparticle surface to promote angiogenesis in hypoxic areas, such as the coronary artery or retinal vessels, the hypoxic state of which contributes to heart disease/myocardial infarction and diabetic retinopathy, respectively. It is understood that a signal protein can also function as a targeting protein. Thus, such a protein can function as either a targeting or a signaling protein, as they bind to a specific receptor, and also initiate/facilitate a certain biological response (e.g., in these cases inflammation and angiogenesis.)

As used herein, the term “targeting enzyme” refers to a targeting protein, including matrix metalloproteinases, such as MMP-1, MMP-9, and MMP-3, which can target different families of collagen in extracellular matrix. Collagen is a common constituent of connective tissue throughout the body. It also has pathological relevance. In cancer, the fibrous extracellular matrix and tumor interstitium is dense with collagen, and thus can serve as a dense barrier to adequate, homogenous drug delivery in chemotherapeutic regimens. In one aspect, targeting enzymes that degrade this collagen-containing matrix can be conjugated to a solid lipid nanoparticle surface, in either singlet (e.g., MMP-10 only) or combined (e.g., MMP-9 and MMP-2 co-functionalization) configurations, to optimize/facilitate drug delivery to tumor cores to create homogenous delivery. It is understood that the use of a targeting enzyme can be can be combined with the use of a targeting protein/antibody in order to specifically target a tumor antigen and degrade its fibrous surroundings with a targeting enzyme.

As used herein, the term “subject” means any target of administration. The subject can be an animal, for example, a mammal. In a further example, the subject can be a human. In an even further example, the subject can be a cell.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

B. Solid Lipid Nanoparticles

In one aspect, the composition of the invention is one or more solid lipid nanoparticle comprising a neutral lipid and a first functionalized polymer, wherein the solid lipid nanoparticle has an interior, an exterior, and a diameter; wherein the first functionalized polymer comprises a polymer having at least one ionic or ionizable moiety, or a copolymer thereof, or mixture thereof; wherein at least a portion of the first functionalized polymer is at the exterior of the solid lipid nanoparticle; and wherein the diameter of the solid lipid nanoparticle is from about 10 nm to about 1,000 nm.

In one aspect, the interior of the solid lipid nanoparticle of the invention refers to the lipid core. The lipid can have a generally spherical or droplet shape with a surface. In one aspect, the first functionalized polymer can be located at the surface. In a further aspect, at least a portion of the first functionalized polymer can be located at the surface. In a further aspect, at least a portion of the first functionalized polymer can be embedded in the lipid.

In one aspect, the exterior of the solid lipid nanoparticle of the invention refers to the volume immediately surrounding the solid lipid nanoparticle. Generally, this volume includes the surface of the lipid and the volume extending outward from the lipid. In one aspect, the first functionalized polymer is at the exterior of the solid lipid nanoparticle.

In one aspect, the diameter of a solid lipid nanoparticle of the invention refers to double the “hydrodynamic radius” of the particle (z, expressed normally in nanometers). The hydrodynamic radius is the effective size of the molecule as detected by its diffusion, derived from the Stokes-Einstein equation. This is the value reported by Dynamic Light Scattering. It is understood that one or more solid lipid nanoparticles can have an average diameter measurement, which can be also referred to as an average particle size.

Solid lipid nanoparticles bearing various surface functionalities can be prepared using a single-step process. Alternately, a multi-step process can be employed to prepare SLN with either mixed or layered surfaces. By using appropriate surface chemistry, trans-endothelial transport of albumin, for example, can be enhanced. Magnetic resonance imaging (MRI) studies show that functionalized SLN can transport an impermeable molecule such as Gadolinium-DTPA across the blood-brain-barrier (BBB) and into brain tissue. Accordingly, functionalized SLN can be used as carrier systems for the delivery of therapeutics and imaging agents to the central nervous system (CNS).

In a further aspect, the composition of the invention is a solid lipid nanoparticle comprising a neutral lipid and a polyether; wherein the solid lipid nanoparticle has an interior, an exterior, and a diameter; wherein at least a portion of the polyether is at the exterior of the solid lipid nanoparticle; and wherein the diameter of the solid lipid nanoparticle is from about 10 nm to about 1,000 nm. In a further aspect, the solid lipid nanoparticle can further comprise at least one of a biologically active agent, a pharmaceutically active agent, a magnetically active agent, or an imaging agent, or a mixture thereof. In a further aspect, a functionalized polymer can also be present. In a further aspect, a functionalized polymer can be absent. In a further aspect, the solid lipid nanoparticle can further comprise at least one of a targeting protein, a signal protein, a targeting enzyme, or an antibody, or a mixture thereof.

Typically, however, the solid lipid nanoparticles of the invention can comprise any of the lipids and polymers of the invention, can be used in any of the methods of the invention, and can be used in any of the applications of the invention.

1. Lipids

Typically, lipids include fats and fat-derived materials that are relatively insoluble in water but soluble in organic solvents, are related either actually or potentially to fatty acid esters, fatty alcohols, sterols, waxes, or the like, and are utilizable by the animal organism. Lipids are one of the chief structural components of living cells. As an example, fats are glyceryl esters of higher fatty acids. In one aspect, the solid lipid nanoparticles of the invention comprise a neutral lipid. That is, for example, the lipid can have substantially no ionic charge. In one aspect, the lipid can be substantially free of charged lipid moieties. For example, the lipid can be substantially free of phospholipid moieties. In a further aspect, the solid lipid nanoparticles of the invention comprise fatty acid glyceryl esters. In a further aspect, the solid lipid nanoparticles of the invention comprise a polymeric lipid. For example, the lipid can be esterified poly(acrylic acid) or esterified poly(vinyl alcohol).

By “solid,” it is meant that at least a portion of the solid lipid nanoparticles of the invention are solid at room temperature and atmospheric pressure. However, it is understood that the solid lipid nanoparticles of the invention can include portions of liquid lipid and/or entrapped solvent.

In one aspect, the lipid can comprise a monoglyceride, diglyceride, or triglyceride of at least one C₄ to C₂₄ carboxylic acid. The carboxylic acid can be saturated or unsaturated and can be branched or unbranched. For example, the lipid can be a monoglyceride of a C₄, C₅, C₆, C₇, C₈, C₉, C₁₀, C₁₁, C₁₂ C₁₃, C₁₄, C₁₅, C₁₆, C₁₇, C₁₈, C₁₉, C₂₀, C₂₁, C₂₂, C₂₃, or C₂₄ carboxylic acid. The carboxylic acid can be saturated or unsaturated and branched or unbranched. The carboxylic acid can be covalently linked to any one of the three glycerol hydroxyl groups. In another example, the lipid can be a diglyceride of C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ carboxylic acids. The two carboxylic acids can be the same or different, and the carboxylic acids can be covalently linked to any two of the three glycerol hydroxyl groups. In a further example, the lipid can be a triglyceride of C₄, C₅, C₆, C₇, C₈, C₉, CIO, Cl , C₁₂, C₁₃, C₁₄, C₁₅, C₁₆, C₁₇, C₁₈, C₁₉, C₂₀, C₂₁, C₂₂, C₂₃, or C₂₄ carboxylic acids. The three carboxylic acids can be the same, two of the carboxylic acid can be the same, or all three can be different. That is, in one aspect, the triglyceride can comprise two fatty acids having the same chain length or can comprise three fatty acids having the same chain length.

In a further aspect, the lipid can comprise a triglyceride of at least one saturated, even-numbered, unbranched natural fatty acid with a chain length of C₈ to C₁₈. For example, the lipid can be a triglyceride of C₈, C₁₀, C₁₂, C₁₄, C₁₆, or C₁₈ carboxylic acids. The three carboxylic acids can be the same, two of the carboxylic acid can be the same, or all three can be different.

In a further aspect, the lipid can comprise a blend of triglycerides of saturated even-numbered, unbranched natural fatty acids with a chain length of C₈ to C₁₈. For example, the lipid can be a blend of triglycerides, each triglyceride of C₈, C₁₀, C₁₂, C₁₄, C₁₆, or C₁₈ carboxylic acids. For each triglyceride in the blend, the three carboxylic acids can be the same, two of the carboxylic acid can be the same, or all three can be different.

In a further aspect, the lipid can comprise a blend of monoglycerides, diglycerides, and triglycerides. The carboxylic acids of each monoglyceride, diglyceride, or triglyceride can be saturated or unsaturated, can be branched or unbranched, and can be a C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ carboxylic acid. In a further aspect, the lipid can comprise a blend of monoglycerides, diglycerides, and triglycerides of saturated even-numbered, unbranched natural fatty acids with a chain length of C₈ to C₁₈. For example, the lipid can be a blend of triglycerides, each triglyceride of C₈, C₁₀, C₁₂, C₁₄, C₁₆, or C₁₈ carboxylic acids.

In a further aspect, the lipid can comprise a triglyceride of palmitic acid, oleic acid, and/or stearic acid. That is, each carboxylic acid of the triglyceride can be palmitic acid, oleic acid, or stearic acid. For each triglyceride in the blend, the three carboxylic acids can be the same, two of the carboxylic acid can be the same, or all three can be different. In a further aspect, the lipid can comprise a triglyceride of palmitic acid, oleic acid, and stearic acid.

In an even further aspect, the lipid can comprise a blend of triglycerides as commercially available under the brand name SOFTISAN®. For example, the lipid can comprise the commercially available SOFTISAN® 100, SOFTISAN®133, SOFTISAN®134, SOFTISAN®138, SOFTISAN® 142, SOFTISAN® 154, or a blend thereof. In a still further aspect, the lipid can comprise a blend of triglycerides as commercially available under the brand name WITEPSOL H35®, and SOFTISAN 133®, SOFTISAN 134®, SOFTISAN 138®, SOFTISAN 378®, SOFTISAN 601®, and/or SOFTISAN 767®.

In a further aspect, the solid lipid nanoparticles of the invention comprise a polymeric lipid. In one aspect, the lipid can be poly(acrylic acid) wholly or partially esterified with one or more alcohols. In one aspect, less than all of the acrylic acid residues are esterified. In a further aspect, substantially all of the acrylic acid residues are esterified. The polymer can be a homopolymer or a copolymer. In one aspect, the lipid can comprise at least one C₄ to C₂₄ alcohol. In one aspect, the alcohol can be saturated or unsaturated, can be branched or unbranched, and can be substituted or unsubstituted. For example, the alcohol can be a C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ alcohol. In a further aspect, the alcohol can comprise at least one saturated, even-numbered, unbranched alcohol with a chain length of C₈ to C₁₈. For example, the alcohol can be a C₈, C₁₀, C₁₂, C₁₄, C₁₆, or C₁₈ alcohol. The alcohols at each acrylic acid residue can be the same or can be different.

In one aspect, the lipid can be poly(vinyl alcohol) wholly or partially esterified with one or more carboxylic acids. In one aspect, less than all of the vinyl alcohol residues are esterified. In a further aspect, substantially all of the vinyl alcohol residues are esterified. The polymer can be a homopolymer or a copolymer. In one aspect, the lipid can comprise at least one C₄ to C₂₄ carboxylic acid. In one aspect, the carboxylic acid can be saturated or unsaturated, can be branched or unbranched, and can be substituted or unsubstituted. For example, the carboxylic acid can be a C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ carboxylic acid. In a further aspect, the carboxylic acid can comprise at least one saturated, even-numbered, unbranched carboxylic acid with a chain length of C₈ to C₁₈. For example, the carboxylic acid can be a C₈, C₁₀, C₁₂, C₁₄, C₁₆, or C₁₈ alcohol. The carboxylic acid at each vinyl alcohol residue can be the same or can be different.

It is understood that the lipids of the invention can be used in combination with the compositions of the invention, methods of the invention, products of the invention, and applications of the invention.

2. Functionalized Polymers

Typically, the solid lipid nanoparticles of the invention can comprise a polymer having functionality of at least one ionic or ionizable moiety. Without wishing to be bound by theory, it is believed that the first functionalized polymer comprises a layer at the exterior of the solid lipid nanoparticle. In one aspect, at least a portion of the functionalized polymers of the invention can be at the exterior of the solid lipid nanoparticle. In this aspect, at least a portion of the functionalized polymers of the invention are surrounding the lipid. In a further aspect, at least a portion of the functionalized polymers of the invention can be embedded in the interior of the solid lipid nanoparticle, that is, in the lipid. In a further aspect, the functionalized polymers of the invention can be surrounding the lipid. In a further aspect, at least one ionic or ionizable moiety is at the exterior of the solid lipid nanoparticle.

In one aspect, the polymer can be a mixture of two or more polymers. In a further aspect, the polymer can be one or more copolymers, including alternating, block, or graft copolymers.

In one aspect, the polymer can comprise a polymer comprising residues of a monomer having at least one ionic or ionizable pendant group. That is, the functionalized polymer of the invention contains the ionic or ionizable group at each or substantially each monomer residue. In a further aspect, the functionalized polymer of the invention contains the ionic or ionizable group at less than each monomer residue.

In one aspect, the functionalized polymers of the invention can have ionic groups or moieties. Ionic groups can have a positive or negative charge and can be singly or multiply charged. Examples of ionic groups include, without limitation, carboxylate, sulfonate, phosphonate, and ammonium groups. Further examples of ionic moieties include, without limitation, phenols/phenoxides and primary, secondary and tertiary amines/ammonium salts.

In one aspect, the functionalized polymers of the invention can have ionizable groups. Ionizable groups can provide an ionic group by gain or loss of an ionic species, for example, a hydrogen ion. Ionizable groups can gain or lose an ionic species to have a positive or negative charge and can then be singly or multiply charged. Examples of ionizable groups include, without limitation, carboxylic, sulfonic, phosphonic, phosphoric, and amine groups.

In one aspect, the first functionalized polymer can be a polymer having at least one ionic or ionizable pendant group. In a further aspect, the first functionalized polymer can be a polymer having at least one ionic or ionizable moiety in the polymer backbone. For example, the at least one ionic or ionizable pendant group can be positively charged or Lewis acidic. In another example, the at least one ionic or ionizable pendant group can be negatively charged or Lewis basic. It is also understood that, in one aspect, the functionalized polymers of the invention can be zwitterionic. In one aspect, the at least one ionic or ionizable pendant group can be a hydroxyl group; an alkoxy salt; a mono-, di-, or tri-substituted amino group; an ammonium salt; a carboxylic acid group, a carboxy group; a sulfonic acid group, a sulfonate salt, or a combination thereof.

In one aspect, the first functionalized polymer comprises, for example, a mono-, di-, or tri-substituted amino group or an ammonium salt; poly(acrylic acid); poly(styrene sulfonate); poly-L-lysine; a copolymer thereof; or a mixture thereof. It is also understood that the first functionalized polymer can be any ionic or ionizable polymer or copolymer known to those of skill in the art including polymers and copolymers of, for example, polyglycine, polyethylene glycol, heparin, hydroxypropylmethylcellulose, hydroxyethylcellulose, hydroxypropylcellulose, polyvinylpyrrolidone, polyvinyl alcohol, methacrylic acid copolymers, ethyl acrylate-methyl methacrylate copolymers, and mixtures thereof. In a further aspect, the first functionalized polymer can be poly(glycolic acid), poly(lactic acid), or copolymers thereof, such as poly(D,L-lactide-co-glycolide), or mixtures thereof.

In a further aspect, the first functionalized polymer can further comprise a polyether, for example, a polyoxyalkane, such as polyoxymethylene, polyethylene glycol, polytrimethylene glycol, or polybutylene glycol; a polycellulosic material; heparin; an antibody-PEG-MALS conjugate; or a copolymer or mixture thereof.

In a further aspect, the functionalized polymer can further comprise a biologically active agent. That is, the functionalized polymer can be a biologically active agent, or the functionalized polymer can be covalently or noncovalently bound or associated with a biologically active agent.

In a further aspect, the functionalized polymer can further comprise a pharmaceutically active agent. That is, the functionalized polymer can be a pharmaceutically active agent, or the functionalized polymer can be covalently or noncovalently bound or associated with a pharmaceutically active agent.

It is understood that the functionalized polymers of the invention can be used in combination with the compositions of the invention, methods of the invention, products of the invention, and applications of the invention.

3. Layered Structure

Typically, the solid lipid nanoparticles of the invention can comprise a layered structure. By “layered” it is meant that, in one aspect, the first functionalized polymer typically substantially surrounds the lipid at the exterior of the solid lipid nanoparticle. In a further aspect, a second functionalized polymer can substantially surround the first functionalized polymer. In a further aspect, a third functionalized polymer can substantially surround the second functionalized polymer. In further aspects, further functionalized polymers can substantially surround the third functionalized polymer. That is, each functionalized polymer can comprise a layer surrounding the lipid of the solid lipid nanoparticles of the invention.

In one aspect, the addition of further layers is accompanied by significant increase in average particles size of the solid lipid nanoparticles of the invention. In a further aspect, the addition of further layers is not accompanied by significant increase in average particles size of the solid lipid nanoparticles of the invention. Typically, depending on the pH of the SLN suspension, the packing of the layers can be affected, and this can lead to a small or substantial increase in SLN size. For example, if the functionalized polymers bear one or more ionized groups, repulsions between like-charged moieties provide a larger functionalized layer and, therefore, a larger hydrodynamic volume of the resultant solid lipid nanoparticle.

In one aspect, a first functionalized polymer can be further functionalized with a second functionalized polymer, a second functionalized polymer can be further functionalized with a third functionalized polymer, a third functionalized polymer can be further functionalized with a fourth functionalized polymer, a fourth functionalized polymer can be further functionalized with a fifth functionalized polymer, and so on. In various aspects, the exterior of the functionalized solid lipid nanoparticles of the invention comprises the outermost functionalized polymer layer.

Typically, each further functionalization with a further functionalized polymer can be performed by adding the further functionalized polymer to the functionalized solid lipid nanoparticles of the invention. In one aspect, a further functionalized polymer is selected to be complementary to the functionalized solid lipid nanoparticles of the invention. By complementary, it is meant that the further functionalized polymer can form a noncovalent or covalent bond with the exterior of the functionalized solid lipid nanoparticles of the invention. In one aspect, the exterior of the functionalized solid lipid nanoparticles of the invention comprises the outermost functionalized polymer layer and, therefore, can bear the functionality of the outermost functionalized polymer layer.

In one aspect, the solid lipid nanoparticle can have two layers. In this aspect, the second layer can be held in a position surrounding the first layer by a bond between the functionalized polymer of the first layer and the complementary functionalized polymer of the second layer. In a further aspect, the bond can be covalent, noncovalent, hydrogen bonding, hydrophobic interactions, hydrophilic interactions, or a mixture thereof. In a further aspect, the bond can be noncovalent, for example ionic: the exterior of the functionalized solid lipid nanoparticles of the invention can comprise a first functionalized polymer comprising a negatively charged polymer, for example poly(styrene sulfonate); and the second functionalized polymer can comprise a positively charged polymer, for example poly-L-lysine.

In a further example, the solid lipid nanoparticle can have three layers. In this aspect, the third layer can be held in a position surrounding the second layer by a bond between the functionalized polymer of the second layer and the complementary functionalized polymer of the third layer. In a further aspect, the bond can be covalent, noncovalent, hydrogen bonding, hydrophobic interactions, hydrophilic interactions, or a mixture thereof. In one aspect, the bond can be noncovalent, for example ionic; the exterior of the functionalized solid lipid nanoparticles of the invention can comprise a first functionalized polymer comprising a negatively charged polymer, for example poly(styrene sulfonate); a second functionalized polymer comprising a positively charged polymer, for example poly-L-lysine; and a third functionalized polymer comprising heparin. Exemplary functionalized polymers that can be used to prepare SLN surface functionality are shown in Table 1.

TABLE 1
SLN SURFACE FUNCTIONALITY

| | Positively | Hydrophilic- | |
|---|---|---|---|
| Negatively Charge | Charged | Neutral | Bioactive |
| Poly(acrylic acid) | Poly-L-Lysine | Polyethylene | Heparin |
| (PAA) | (PLys) | glycol (PEG) | (Hep) |
| Poly(styrene | | Poly(vinyl | |
| sulfonate) (PSS) | | alcohol) (PVA) | |

It is understood that a functionalized polymer can be added to the solid lipid nanoparticles of the invention, for example, as a solid, as a liquid, as a solution, as a suspension, as an emulsion, or a mixture thereof.

It is understood that any number of layers can be assembled around the solid lipid nanoparticles of the invention. For example, there can be one, two, three, four, five, six, seven, eight, nine, or ten layers. In further aspects, there can be more than ten layers.

It is also understood that the layers of the invention can be used in combination with the compositions of the invention, methods of the invention, products of the invention, and applications of the invention.

a. First Layer

Typically, in one aspect, the first functionalized polymer can be a polymer having at least one ionic or ionizable moiety, for example, a pendant group or moiety in the polymer backbone. In one aspect, the at least one ionic or ionizable pendant group is positively charged or Lewis acidic. In a further aspect, the at least one ionic or ionizable pendant group is negatively charged or Lewis basic. It is understood that more than one first functionalized polymer can be used in connection with the solid lipid nanoparticles of the invention.

In one aspect, the at least one ionic or ionizable moiety can be a hydroxyl group; an alkoxy salt; a mono-, di-, or tri-substituted amino group; an ammonium salt; a carboxylic acid group, a carboxy group; a sulfonic acid group, a sulfonate salt, or a combination thereof. In further aspects, surface first functional polymer layers contemplated for use in the present system and methods include polymers and copolymers of, for example, poly(acrylic acid), poly-L-lysine, polyglycine, polyethylene glycol, heparin, hydroxypropylmethylcellulose, hydroxyethylcellulose, hydroxypropylcellulose, polyvinylpyrrolidone, polyvinyl alcohol, methacrylic acid copolymers, ethyl acrylate-methyl methacrylate copolymers, and mixtures thereof. In a further aspect, the first functionalized polymer can be poly(acrylic acid) or poly(styrene sulfonate) or poly-L-lysine. In an even further aspect, the first functionalized polymer can be poly(allyl amine), poly(histidine), polyethyleneimine (PEI), or a mixture thereof. These polymers are known to those of skill in the art. For instance, polyethyleneimine is a positively charged polymer that can be used as a transfection reagent.

In one aspect, the first functionalized polymer further comprises a polyether. That is, the functionalized polymer can be covalently or noncovalently bound or associated with a polyether. The polyether can be, for example, heparin, polyoxymethylene, polyethylene glycol, polytrimethylene glycol, polybutylene glycol, or an antibody-PEG-MALS conjugate, or a copolymer thereof, or a mixture thereof.

In one aspect, the first functionalized polymer can further comprise a biologically active agent. That is, the functionalized polymer can be a biologically active agent, or the functionalized polymer can be covalently or noncovalently bound or associated with a biologically active agent.

In a further aspect, the first functionalized polymer can further comprise a pharmaceutically active agent. That is, the functionalized polymer can be a pharmaceutically active agent, or the functionalized polymer can be covalently or noncovalently bound or associated with a pharmaceutically active agent.

It is understood that the first functionalized polymers of the invention can be used in combination with the compositions of the invention, methods of the invention, products of the invention, and applications of the invention.

b. Second Layer

In a further aspect, the solid lipid nanoparticles of the invention can be further functionalized with a second functionalized polymer. It is understood that more than one second functionalized polymer can be used in connection with the solid lipid nanoparticles of the invention. In one aspect, a second functionalized polymer can substantially surround the first functionalized polymer. The second functionalized polymer can be the same as or different than the first functionalized polymer, but, in one aspect, the second functionalized polymer is complementary to the first functionalized polymer. That is, in this aspect, the second functionalized polymer can form a covalent or noncovalent, for example ionic, bond with the first functionalized polymer.

In one aspect, the second functionalized polymer can further comprise a polyether. That is, the functionalized polymer can be covalently or noncovalently bound or associated with a polyether. In a further aspect, the second functionalized polymer can further comprise a biologically active agent. That is, the functionalized polymer can be a biologically active agent, or the functionalized polymer can be covalently or noncovalently bound or associated with a biologically active agent. In a further aspect, the second functionalized polymer can further comprise a pharmaceutically active agent. That is, the functionalized polymer can be a pharmaceutically active agent, or the functionalized polymer can be covalently or noncovalently bound or associated with a pharmaceutically active agent.

It is understood that the second functionalized polymers of the invention can be used in combination with the compositions of the invention, methods of the invention, products of the invention, and applications of the invention. It is also understood that the second functionalized polymers can be absent from the solid lipid nanoparticles of the invention.

C. Third Layer

In a further aspect, the solid lipid nanoparticles of the invention can be further functionalized with a third functionalized polymer. It is understood that more than one third functionalized polymer can be used in connection with the solid lipid nanoparticles of the invention. In one aspect, a third functionalized polymer can substantially surround the second functionalized polymer. The third functionalized polymer can be the same as or different than the first functionalized polymer or the second functionalized polymer, but, in one aspect, the third functionalized polymer is complementary to the second functionalized polymer. That is, in this aspect, the third functionalized polymer can form a covalent or noncovalent, for example ionic, bond with the second functionalized polymer.

In one aspect, the third functionalized polymer can further comprise a polyether. That is, the functionalized polymer can be covalently or noncovalently bound or associated with a polyether. In a further aspect, the third functionalized polymer can further comprise a biologically active agent. That is, the functionalized polymer can be a biologically active agent, or the functionalized polymer can be covalently or noncovalently bound or associated with a biologically active agent. In a further aspect, the third functionalized polymer can further comprise a pharmaceutically active agent. That is, the functionalized polymer can be a pharmaceutically active agent, or the functionalized polymer can be covalently or noncovalently bound or associated with a pharmaceutically active agent.

It is understood that the third functionalized polymers of the invention can be used in combination with the compositions of the invention, methods of the invention, products of the invention, and applications of the invention. It is also understood that the third functionalized polymers can be absent from the solid lipid nanoparticles of the invention.

d. Successive Layers

In a further aspect, the solid lipid nanoparticles of the invention can be further functionalized with one or more successive functionalized polymers. In one aspect, each successive functionalized polymer can substantially surround the third functionalized polymer, the fourth functionalized polymer, the fifth functionalized polymer, and so on. The successive functionalized polymer can be the same as or different than the first functionalized polymer, the second functionalized polymer, or the third functionalized polymer, but, in one aspect, each successive functionalized polymer is complementary to the third functionalized polymer, the fourth functionalized polymer, the fifth functionalized polymer, and so on. That is, in this aspect, the successive functionalized polymer can form a covalent or noncovalent, for example ionic, bond with the third functionalized polymer, the fourth functionalized polymer, the fifth functionalized polymer, and so on.

In one aspect, the successive functionalized polymer can further comprise a polyether. That is, the functionalized polymer can be covalently or noncovalently bound or associated with a polyether. In a further aspect, the successive functionalized polymer can further comprise a biologically active agent. That is, the functionalized polymer can be a biologically active agent, or the functionalized polymer can be covalently or noncovalently bound or associated with a biologically active agent. In a further aspect, the successive functionalized polymer can further comprise a pharmaceutically active agent. That is, the functionalized polymer can be a pharmaceutically active agent, or the functionalized polymer can be covalently or noncovalently bound or associated with a pharmaceutically active agent.

It is understood that the successive functionalized polymers of the invention can be used in combination with the compositions of the invention, methods of the invention, products of the invention, and applications of the invention. It is also understood that the successive functionalized polymers can be absent from the solid lipid nanoparticles of the invention.

4. Surface Active Agents

Typically, surface active agents can be used in connection with the solid lipid nanoparticles of the invention and the functionalized polymers of the invention. In one aspect, surface active agents can include, for example, a polyether, a biologically active agent, a pharmaceutically active agent, a stabilizing agent, or a mixture thereof. In one aspect, each surface active agent can provide a function, for example, the surface active agent can be biologically active, pharmaceutically active, or can suppress immune recognition.

In one aspect, the surface active agents of the invention can be provided at the exterior of the solid lipid nanoparticles of the invention. That is, surface active agent can be covalently or noncovalently bound or associated with any of the functionalized polymers of the invention. It is understood that more than one surface active agent can be used in connection with the solid lipid nanoparticles of the invention. In one aspect, the surface active agents of the invention can exclude surfactants and/or emulsifiers. It is also understood that the surface active agents can be absent from the solid lipid nanoparticles of the invention.

It is understood that the surface active agents of the invention can be used in combination with the compositions of the invention, methods of the invention, products of the invention, and applications of the invention.

a. Polyethers

In one aspect, the surface active agent can be a polyether. In a further aspect, a polyether has a carbon-oxygen-carbon moiety in the polymer backbone. In a further aspect, the polyether has a carbon-oxygen-carbon moiety as a pendant group. The polyether can be any polyether known to those of skill in the art and can include, for example, heparin, polyoxymethylene, polyethylene glycol, poly(1,2-propylene glycol), polytrimethylene glycol, polybutylene glycol, or an antibody-PEG-MALS conjugate, or a copolymer thereof, or a mixture thereof. In one aspect, polyethers can be absent from the solid lipid nanoparticles of the invention.

Polyethers are known to possess resistance to biological systems. That is, polyethers can be unaffected, or less affected, by biological systems, for example immune recognition, than other materials. Accordingly, nanoparticles comprising polyethers, in particular nanoparticles bearing polyethers at the surface, can also have enhanced resistance to biological systems.

It is understood that the polyethers of the invention can be used in combination with the compositions of the invention, methods of the invention, products of the invention, and applications of the invention.

b. Biologically Active Agents

In one aspect, the surface active agent can be a biologically active agent. Typically, the biologically active agent can be any biologically active agent known to those of skill in the art and can include, for example, any of the biologically active agents disclosed herein. In one aspect, the biologically active agent can be a targeting protein, a signal protein, or a targeting enzyme.

It is understood that more than one biologically active agent can be used in connection with the solid lipid nanoparticles of the invention. It is also understood that the biologically active agents can be absent from the solid lipid nanoparticles of the invention.

It is understood that the disclosed biologically active agents can be used in combination with the compositions of the invention, methods of the invention, products of the invention, and applications of the invention.

C. Pharmaceutically Active Agents

In one aspect, the surface active agent can be a pharmaceutically active agent. Typically, the biologically active agent can be any biologically active agent known to those of skill in the art and can include, for example, any of the pharmaceutically active agents disclosed herein.

It is understood that more than one pharmaceutically active agent can be used in connection with the solid lipid nanoparticles of the invention. It is also understood that the pharmaceutically active agents can be absent from the solid lipid nanoparticles of the invention.

It is understood that the pharmaceutically active agents of the invention can be used in combination with the compositions of the invention, methods of the invention, products of the invention, and applications of the invention.

d. Stabilizing Agents

In one aspect, the surface active agent can be a stabilizing agent.

Examples of stabilizing agents that can be used in connection with the invention include, without limitation, a crystalline NP stabilizing agent such as cholesterol, cholesterol-bearing pullulan, polystyrene, the functionalized polymers of the invention, and mixtures thereof.

It is understood that more than one stabilizing agent can be used in connection with the solid lipid nanoparticles of the invention. It is also understood that the stabilizing agents can be absent from the solid lipid nanoparticles of the invention.

It is understood that the stabilizing agents of the invention can be used in combination with the compositions of the invention, methods of the invention, products of the invention, and applications of the invention.

5. Payloads

Typically, payloads can be used in connection with the solid lipid nanoparticles of the invention and the functionalized polymers of the invention. In one aspect, payloads can include, for example, a pharmaceutically active agent, a magnetically active agent, an imaging agent, or a mixture thereof. In one aspect, each payload can provide a function, for example, the surface active agent can be pharmaceutically active, magnetically active, or can provide a detectable response for imaging.

In one aspect, the payloads of the invention can be provided at the interior of the solid lipid nanoparticles of the invention. That is, the disclosed payloads can be encapsulated within the lipid of the solid lipid nanoparticles of the invention. It is understood that more than one payload can be used in connection with the solid lipid nanoparticles of the invention. It is also understood that the payloads can be absent from the solid lipid nanoparticles of the invention.

In one aspect, payloads can include, for example, a delivery package, such as a biologically active agent, a pharmaceutically active agent, a magnetically active agent, an imaging agent, or a mixture thereof.

It is understood that the disclosed payloads can be used in combination with the compositions of the invention, methods of the invention, products of the invention, and applications of the invention.

a. Pharmaceutically Active Agents

In one aspect, the payload can be a pharmaceutically active agent. Typically, the pharmaceutically active agent can be any pharmaceutically active agent known to those of skill in the art and can include, for example, any of the pharmaceutically active agents disclosed herein.

It is understood that more than one pharmaceutically active agent can be used in connection with the solid lipid nanoparticles of the invention. It is also understood that the pharmaceutically active agents can be absent from the solid lipid nanoparticles of the invention.

It is understood that the pharmaceutically active agents of the invention can be used in combination with the compositions of the invention, methods of the invention, products of the invention, and applications of the invention.

b. Magnetically Active Agents

In one aspect, the payload can be a magnetically active agent. Typically, the magnetically active agent can be any magnetically active agent known to those of skill in the art and can include, for example, diamagnetic, paramagnetic, ferromagnetic, and/or ferromagnetic materials. In one aspect, the magnetically active agents of the invention can include particles or clusters of Magnetite, Maghemite, Jacobsite, Trevorite, Magnesioferrite, Pyrrhotite, Greigite, Feroxyhyte, Iron, Nickel, Cobalt, Awaruite, Wairauite, Manganese salts, or mixtures thereof. In a further aspect, the magnetically active agent comprises iron, nickel, or magnetite. In a further aspect, the magnetically active agent comprises magnetite.

It is understood that more than one magnetically active agent can be used in connection with the solid lipid nanoparticles of the invention. It is also understood that the magnetically active agents can be absent from the solid lipid nanoparticles of the invention.

It is also understood that the magnetically active agents of the invention can be used in combination with the compositions of the invention, methods of the invention, products of the invention, and applications of the invention.

Magnetically active agents, for example, paramagnetic iron oxide nanoparticles, are promising tools in biomedical applications. Their principle characteristics include nanoscale feature sizes, the capacity to be controlled by externally applied magnetic fields, T2 relaxation time shortening for use as a contrast enhancement agent in magnetic resonance imaging, and amenability to surface functionalization (See Berry, C. C., and A. S. G. Curtis, 2003, Functionalisation of magnetic nanoparticles for applications in biomedicine, J Phys D Appl Phys 36(13):R198-R206). Drawbacks to clinical usage of paramagnetic nanoparticles include a tendency for nanoparticles to aggregate upon application of the magnetic field (See Bonnemain, B., 1998, Superparamagnetic agents in magnetic resonance imaging: Physicochemical characteristics and clinical applications—A review, J Drug Target 6(3):167-174.), and the need for complex, multistep chemical cross-linking procedures for the functionalization of drugs, proteins, or polymers. The present invention uses a lipid encapsulation strategy for the single-step entrapment of magnetically active agents, for example paramagnetic iron oxide nanoparticles, within a lipid vesicle, the surface of which is water soluble and amenable to surface functionalization of many species.

The techniques of the present invention have application in the development of magnetooptical probes employing fluorophores (e.g., quantum dots or fluorescently-labeled proteins) and magnetically active agents, for example, iron oxide nanoparticles, co-localized in the same matrix such as a SLN for tumor targeting and imaging, which have applications for in vivo imaging of circulation and tissue using MRI and fluorescence imaging techniques such as multiphoton excitation microscopy. Such imaging can be performed to study the pharmacodynamics and pharmacokinetics of a drug in vivo.

The present techniques reduce the aggregation tendencies of magnetically active agents, for example paramagnetic nanoparticles, and facilitate the transition to clinical uses in imaging and drug delivery. Furthermore, the ease of encapsulation provides a vehicle for a variety of therapeutic proteins and drugs by introduction in the aqueous or organic phases prior to phase inversion, without the need for chemical modification of the solid lipid nanoparticle surface. The solid lipid nanoparticles of the invention are capable of transporting magnetically active agents, for example iron oxide nanoparticles, and thus may serve as a useful contrast agent for MRI, which minimizes false signal due to nonspecific nanoparticle agglomeration in vivo. Combined with the ability to surface functionalize the lipid moieties in a single step, the solid lipid nanoparticles of the invention are versatile agents for the specific targeting of tissue.

c. Imaging Agents

In one aspect, the payload can be an imaging agent. Typically, the imaging agent can be any imaging agent known to those of skill in the art and can include, for example, radioconjugate, cytotoxin, cytokine, Gadolinium-DTPA or a quantum dot. In one aspect, the imaging agent comprises Gadolinium-DTPA and iron oxide nanoparticles (magnetite), as specific MRI contrast agents. In a further aspect, the imaging agent comprises at least one near infrared dye, for example near infrared dyes based on a porphyrin and/or a phthalocyanine. See Ghoroghchian et al., Near-infrared-emissive polymersomes: Self-assembled soft matter for in vivo optical imaging, PNAS, 2005, vol. 102, no. 8, 2922-2927. In a further aspect, the imaging agent comprises two or more quantum dots, wherein the two or more quantum dots have different emission wavelengths. It is understood more than one imaging agent can be used in connection with the multimodal applications of the inventions, such as quantum dot—Gd-DTPA—iron oxide nanoparticle co-encapsulated species.

In one aspect, the compositions of the invention and methods can be used to provide a functionalized quantum dot comprising one or more quantum dots encapsulated within the solid lipid nanoparticles of the invention. That is, the solid lipid nanoparticles of the invention can be used to functionalize a quantum dot before administration of the quantum dot to a subject.

Typically, a quantum dot is a photostable color-tunable nanocrystal with a wide absorption spectrum and a narrow emission peak. Quantum dots, also called nanocrystals, are semiconductor crystals with a diameter of a few nanometers and, because of the small size, behave like potential wells that confines electrons in three dimensions to a region on the order of the electrons' de Broglie wavelength in size, that is, a few nanometers in a semiconductor.

Quantum dots (QD) have emerged as a powerful research tool in fluorescence imaging due to their unique optical properties, which include enhanced intensity and photostability compared to organic dyes, and size-tunable emission spectra. Among the many challenges, rapid internalization of QD is particularly important, specifically for imaging cytosolic structures and processes in real time. Current strategies for intracellular delivery of QD rely on the surface functionalization of the QD nanocrystals with internalization peptides (See Akerman, M. E., W. C. W. Chan, et al. (2002) “Nanocrystal targeting in vivo.” Proceedings of the National Academy of Sciences of the United States of America 99(20): 12617-12621; Derfus, A. M., W. C. W. Chan, et al. (2004) “Probing the cytotoxicity of semiconductor quantum dots.” Nano Letters 4(1): 11-18; Watson, A., X. Y. Wu, et al. (2003) “Lighting up cells with quantum dots.” Biotechniques 34(2): 296-+). Surface functionalization involving polymers or peptides can often be time-consuming, requiring different coupling chemistries and steps for introduction of multiple functionalities, and these strategies have the potential of compromising the integrity of the QD nanocrystals, resulting in reduced quantum efficiencies. (See Fan, H. Y., E. W. Leve, et al. (2005) “Surfactant-assisted synthesis of water-soluble and biocompatible semiconductor quantum dot micelles.” Nano Letters 5(4): 645-648). It is notable that conventional methods typically rely upon physical introduction of QD into cells, by membrane perturbing methods such as electroporation and microinjection or by slowly-internalized liposomes. For example, microinjection methods rely on single-cell perturbations with a needle, which is unrealistic for large populations of cells. Electroporation also can have damaging effects on cell membranes (see, e.g., J Gene Med., September 2005;7(9):1235-45).

Even with these functionalization approaches, internalization of QD in cells is in the order of hours. Delivery vehicles with rapid internalization times would be more suitable for time-critical assays, such as neutrophil functional assays or studies of cellular behavior shortly after tissue biopsies. Techniques for increasing the stability of QD in aqueous environments have also been explored (See Dubertret, B., P. Skourides, et al. (2002) “In vivo imaging of quantum dots encapsulated in phospholipid micelles,” Science 298(5599): 1759-1762; Ballou, B., B. C. Lagerholm, et al., (2004) “Noninvasive imaging of quantum dots in mice,” Bioconjugate Chemistry 15(1): 79-86). However, such systems do not completely address the problem of diminished stability of QD in aqueous environments due to oxidative degradation (See Derftis, A. M., W. C. W. Chan, et al. (2004) “Probing the cytotoxicity of semiconductor quantum dots,” Nano Letters 4(1): 11-18). While these conventional techniques may add optical and physical stability to QD, they typically do not address the intracellular delivery issue—none can get into cells unless a physical introduction method is used.

Therefore, stable quantum dot delivery vehicles that protect their contents from aqueous environments without structurally compromising the nanocrystal structure can be highly desirable in biological applications. Such applications can also require bioconjugates readily amenable to surface functionalization. A process for rapid surface functionalization of quantum dots with multiple species such as antibodies, peptides and polymeric species can be a significant advancement towards expanding the utility of QD. Lastly, in fluorescence detection applications, signal amplification is often desired, as in receptor targeting assays. For this reason, a process which yields a delivery vehicle carrying multiple quantum dots can serve as a useful tool in such assays. Also desirable is a method for passive, energy-independent internalization of quantum dots into cells, a method which would result in stable QD being targeted within the cytoplasm to specific subcellular organelles and/or proteins and/or nuclear structures, without being sequestered within endosomes. Ideally, such internalization should be rapid to enable quick studies on cells with short lifetimes (e.g., neutrophils from peripheral blood, 4 hrs maximum).

Functionalized SLN-QD can be prepared by inducing a rapid phase inversion in a binary solvent system, for example N-methylpyrolidinone (NMP) and acetone, containing dissolved lipids and a suspension of QD, by the addition of an aqueous phase. By the incorporation of polyionic or polyionizable species in the aqueous phase, functionalized QD-SLN can be prepared in a single step. Varying the ratio of NMP to acetone allows control over key properties of the SLN-QD, such as size and QD loading. SLN-QD ranging from about 10 nm to about 1000 nm, for example, from about 50 nm to about 400 nm, in diameter can be prepared using this strategy, as measured by photon correlation spectroscopy. Since NMP has a greater affinity for water, increasing NMP volume fraction results in rapid influx of water into the organic phase leading to smaller SLN-QD. By decreasing the NMP volume fraction, a slower influx of water into the organic phase results in a slower packing of the lipid colloid to yield larger nanoparticles. Solid lipid nanoparticles containing multiple QD nanocrystals, bearing anionic (e.g., poly(styrene sulfonate) (PSS)) and cationic (e.g., poly-L-lysine (PLL)) moieties have been produced. The presence of multiple well-dispersed QD in a single SLN was verified by TEM analysis, which also revealed higher loading densities with increasing SLN diameter. A direct consequence of an increased loading density can be signal amplification. The presence of surface functionality in the SLN-QD can be verified by Zeta potential measurements on SLN-QD, which indicated that isoelectric points can corresponded closely with the pKa of the ionizable group. For example, in SLN-QD bearing PSS, the pKa of the surface was around 2 pH units, which is comparable to the pKa of the sulfonic acid groups on the PSS. Similar observations were made in SLN-QD modified to bear PLL, poly(acrylic acid) (PAA), and poly(ethylene glycol) (PEG) moieties. The capability to attach polyvalent functional groups and moieties such as PEG on SLN-QD can have implications in enhancing circulation times (See Klibanov, A. L., K. Maruyama, et al., (1990) “Amphipathic Polyethyleneglycols Effectively Prolong the Circulation Time of Liposomes,” Febs Letters 268(1): 235-237) and targeting QD to mucosal tissue, by secondary chemical modifications using standard protein conjugation chemistries and layer-by-layer electrostatic assembly.

Spectrophotometric and fluorimetric measurements of SLN-QD spectra revealed that the narrow characteristic emission spectra of QD about 580 nm was preserved even upon encapsulation in a functionalized SLN matrix. This observation confirms that the disclosed lipid encapsulation strategy does not negatively affect the fundamental optical properties of quantum dots. An important outcome of encapsulation of QD in a lipid matrix is the enhanced stability of the QD in aqueous environments. When exposed to lactated Ringers buffer, hydrophobic, unencapsulated core-shell quantum dots rapidly aggregated within minutes. In contrast, lipid-encapsulated quantum dots remained monodisperse and stable in an aqueous suspension. Without wishing to be bound by theory, it is believed that monodispersity is a direct result of the steric stabilization conferred by the negatively charged PSS groups on SLN-QD. Furthermore, SLN-QD exhibited no notable changes in optical properties even after 6 months in solution. Labeling of adherent BAEC cultures was performed with PSS-functionalized SLN-QD. Flow cytometric analysis of bovine aortic endothelial cells (BAEC) incubated with SLN-QD for at least 10 minutes indicated rapid loading of a majority of the cell fraction. The rapid uptake of QD-SLN by BAEC cells was confirmed by live cell fluorescence imaging and more notably occurred without any visual damage to cell membrane integrity. Confocal microscopy of SLN-QD incubated BAEC indicated the presence of perinuclear SLN-QD labeling in a majority of the cells. A diffuse, punctuate staining pattern was observed as opposed to a clumped, granular appearance, suggesting that SLN-QD may not be localized within endocytic vesicles, but could be freely dispersed in the cytoplasm. These observations indicate that SLN-QD interaction with cell membrane is via lipid-mediated events and not through a receptor mediated process. The disclosed strategy can serve as a useful technique for the encapsulation of quantum dots for intracellular delivery of multi-functional bioconjugates, while protecting quantum dot properties by isolation from the cellular environment.

The synthesis of functionalized SLN-QD can be accomplished in a single step without surfactants. The sizes of the SLN-QD can be highly tunable with simple adjustments in solvent polarity. SLN-QD can be rapidly loaded into cells relative to existing quantum dot delivery systems, which require hours of incubation. One application of the disclosed system not readily achievable with existing quantum dot strategies is signal amplification of weak antigens. Other applications of this technology include, for example, intracellular delivery of functionalized quantum dots for live cell labeling of organelles or filaments, and co-encapsulation of hydrophobic drugs for pharmacokinetic studies.

It is understood that the disclosed imaging agents can be used in combination with the compositions of the invention, methods of the invention, products of the invention, and applications of the invention.

6. Properties

In one aspect, the solid lipid nanoparticles of the invention can be provided substantially free of surfactants or emulsifying agents. However, it is understood that surfactants or emulsifying agents can be used in connection with the solid lipid nanoparticles of the invention.

In a further aspect, the solid lipid nanoparticles of the invention can form a stable dispersion in water.

In further aspects, the solid lipid nanoparticles of the invention can have a particle size, a melting temperature, and a zeta potential.

a. Particle Size

Typically, the particle size of the solid lipid nanoparticles of the invention can be a function of the binary solvent system selected and can be modulated by this selection. The particle size can also be a function of the concentration and temperature of the binary solvent, the concentration, temperature, and composition of the aqueous phase.

In one aspect, the solid lipid nanoparticles of the invention can be provided having a particle size of from about 10 nm to about 1000 nm. In a further aspect, the particle size can be from about 10 nm to about 900 nm, from about 10 nm to about 800 nm, from about 10 nm to about 700 nm, from about 10 nm to about 600 nm, from about 10 nm to about 500 nm, from about 10 nm to about 400 nm, from about 10 nm to about 300 nm, from about 10 nm to about 200 nm, or from about 10 nm to about 100 nm.

In a yet further aspect, the particle size can be from about 25 nm to about 400 nm, from about 25 nm to about 300 nm, from about 25 nm to about 200 nm, from about 25 nm to about 100 nm, or from about 25 nm to about 50 nm. In an even further aspect, the particle size can be from about 50 nm to about 300 nm, from about 50 nm to about 200 nm, or from about 50 nm to about 100 nm. In a still further aspect, the particle size can be from about 200 nm to about 300 nm, from about 220 nm to about 280 nm, from about 240 nm to about 260 nm, from about 200 nm to about 280 nm, or from about 220 nm to about 300 nm.

b. Melting Temperature

Typically, the melting temperature of the solid lipid nanoparticles of the invention can be a function of the lipid selected and can be modulated by this selection. For example, solid lipid nanoparticles can be prepared from a lipid blend provided by selecting a first lipid having a first melting point of a first temperature and combining the first lipid with a second lipid having a second melting point of a second temperature. The melting temperature of the blend provided can be approximately the weighted average of the ratio of the masses of the first and second lipids. That is, by selecting and blending lipids of known melting temperatures, one of skill in the art can modulate the melting temperature of the blend, and nanoparticles, thereby provided.

In one aspect, the melting temperature can be from about 25° C. to about 100° C., for example, from about 25° C. to about 50° C., from about 30° C. to about 80° C., from about 50° C. to about 75° C., or from about 30° C. to about 40° C. In a further aspect, the melting temperature can be from about 37° C. to about 50° C., for example, from about 52° C. to about 54° C., about 38° C., about 39° C., about 40° C., about 41° C., about 42° C., about 43° C., about 44° C., about 45° C., about 46° C., about 47° C., about 48° C., about 49° C., or about 50° C. In a further aspect, the melting temperature can be from about 32° C. to about 37° C., for example, about 33° C., about 34° C., about 35° C., or about 36° C.

In a further aspect, the solid lipid nanoparticles of the invention can have more than one melting temperature. That is, a solid lipid nanoparticle mixture can be provided by combining at least one solid lipid nanoparticle comprising a first lipid having a first melting point of a first temperature with at least one solid lipid nanoparticle comprising a second lipid having a second melting point of a second temperature. At least a portion of the resultant mixture can then melt at the first temperature and at least a portion can then melt at the second temperature.

It is understood that lipid blends can be used to prepare nanoparticle mixtures.

c. Zeta Potential

Zeta potential can be an indicator of particle surface charge, which can be used to predict and control the stability of colloidal suspensions or emulsions. In general, the greater the zeta potential, the more likely a suspension is to be stable because the charged particles repel one another and thus overcome the natural tendency to aggregate. Zeta potential can also be a controlling parameter in processes such as adhesion, surface coating, filtration, lubrication, and corrosion.

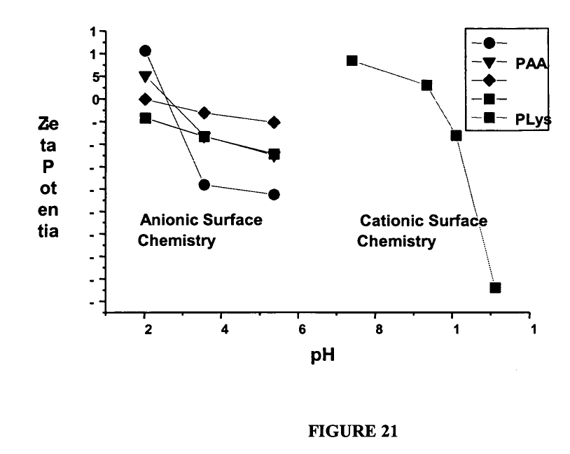

A graph illustrating the relationship between zeta potential and pH for the solid lipid nanoparticles of the invention is shown in FIG. 21.

In one aspect, the zeta potential of the solid lipid nanoparticles of the invention can be a function of the pH of the suspension as well as the functionalized polymer(s) selected and can be modulated by this selection. In a further aspect, the zeta potential of the solid lipid nanoparticles of the invention can be sufficient to maintain a stable suspension or dispersion.

In one aspect, the zeta potential can be positive. For example, the zeta potential can be greater than about 5 mV, greater than about 10 mV, greater than about 15 mV, greater than about 20 mV, greater than about 25 mV, greater than about 30 mV, greater than about 35 mV, greater than about 40 mV, greater than about 45 mV, greater than about 50 mV, greater than about 75 mV, or greater than about 100 mV.

In a further aspect, the zeta potential can be negative. For example, the zeta potential can be more negative than about −5 mV, more negative than about −10 mV, more negative than about −15 mV, more negative than about −20 mV, more negative than about −25 mV, more negative than about −30 mV, more negative than about −35 mV, more negative than about −40 mV, more negative than about −45 mV, more negative than about −50 mV, more negative than about −75 mV, or more negative than about −100 mV.

In a further aspect, the zeta potential of the solid lipid nanoparticles of the invention can be correlated with the pKa of the ionic or ionizable moieties at the surface of the solid lipid nanoparticles. Consequently, at a suspension pH of approximately the pKa of the ionic or ionizable moieties at the surface of a nanoparticle, an inversion of zeta potential can be observed. That is, as the pH of the suspension approaches and passes the pKa of the ionic or ionizable moieties at the surface of a nanoparticle, the zeta potential can change polarity from positive to negative or negative to positive. This inversion point can also be referred to as the isoelectric point of the surface functionalized polymer and, therefore, of the solid lipid nanoparticle.

C. Methods of Making Solid Lipid Nanoparticles

In one aspect, the solid lipid nanoparticles of the invention can be prepared by providing an organic phase comprising a binary solvent system and a neutral lipid; providing an aqueous phase comprising water and at least one first functionalized polymer having at least one ionic or ionizable moiety; and combining the organic phase and the aqueous phase. The organic phase, or a portion of the organic phase, can be optionally removed, thereby providing the solid lipid nanoparticles as an aqueous suspension. Typically, the compositions of the invention and methods of the invention can be used to prepare the solid lipid nanoparticles; however, it is understood that any compositions and methods known to those of skill in the art can also be used in connection with the solid lipid nanoparticles of the invention.

SLN bearing different surface functionalities can be prepared by a phase inversion process. Specifically, SLN components, for example a neutral lipid, can be dissolved in a solvent system, for example, a binary solvent system such as N-methylpyrolidinone (NMP)-acetone, tetrahydrofuran (THF)-acetone, or dimethylformamide (DMF)-acetone. An aqueous solution containing functionalized polymer, also referred to as surface functional moieties, such as poly(acrylic acid), poly-L-lysine, polyglycine, polyethylene glycol, heparin, hydroxypropylmethylcellulose, hydroxyethylcellulose, hydroxypropylcellulose, polyvinylpyrrolidone, polyvinyl alcohol, methacrylic acid copolymers, ethyl acrylate-methyl methacrylate copolymers, or mixtures thereof, can then be prepared and added to the solution. Due to intermolecular interactions, a layer of surface functional moieties—also referred to as the surface functional layer—surrounds the SLN. Typically, the intermolecular interactions are noncovalent and can be, for example, due to hydrophilic, hydrophobic, hydrogen bonding, or ionic interactions. The step of adding an aqueous solution containing surface functional moieties can be repeated one or more times to produce a mixed surface functional layers or to produce surface functional layers in successive coatings around the SLN.

Surface functional moieties can be selected so as to achieve a resultant particle bearing a specific surface functional layer. Specifically, a negatively charged surface functional layer can be prepared by selecting a negatively charged surface functional moiety, for example, a functionalized polymer such as poly(acrylic acid). Also, a positively charged surface functional layer can be prepared by selecting a positively charged surface functional moiety, for example, a functionalized polymer such as poly-L-lysine. A hydrophilic surface functional layer can be prepared by selecting a hydrophilic surface functional moiety, for example, a functionalized polymer such as polyethylene glycol. A bioactive surface functional layer can be prepared by selecting a bioactive surface functional moiety, for example, heparin.

Due to the stabilization effect of the functional moieties, the SLN can be produced in the absence of surfactants. Further, the use of solvent obviates any melting or solidification step normally associated with the preparation of SLN. The procedure also allows for simultaneous encapsulation of both hydrophilic and hydrophobic small molecules and proteins. The unique nature of SLN surface properties is borne out by their distinct charge characteristics. For example, the isoelectric points of the various SLN that can be prepared according to the present method correspond to the pKa of the functional groups present in the surface functional moieties used in the preparation.

In one aspect, the solid lipid nanoparticles of the invention can be provided substantially free of surfactants or emulsifying agents. However, it is understood that surfactants or emulsifying agents can be used in connection with the solid lipid nanoparticles of the invention.

In a further aspect, the organic phase can be separated from the aqueous phase subsequent to preparation of the solid lipid nanoparticles. Because formation of the solid lipid nanoparticles can occur substantially instantaneously during the combining step by a phase inversion process, the organic phase can be optionally removed after the combining step. In a further aspect, at least a portion of the organic phase can be separated from the aqueous phase. That is, one component of the binary solvent system of the organic phase can be more miscible with the aqueous phase than the other component(s) of the binary solvent system. In such a case, at least a portion of the more miscible component can remain with the aqueous phase when the organic phase is separated. Separation can occur by any means of separation known to those of skill in the art, for example, evaporation, phase separation, spray drying, distillation, or the like.

1. Methods of Modulating Particle Size

In one aspect, the methods of the invention can comprise the steps of selecting a binary solvent system; dissolving a neutral lipid in the binary solvent system, thereby producing an organic phase; providing an aqueous phase comprising a first functionalized polymer; and combining the organic phase and the aqueous phase, thereby producing a substantially monodisperse solid lipid nanoparticle suspension.

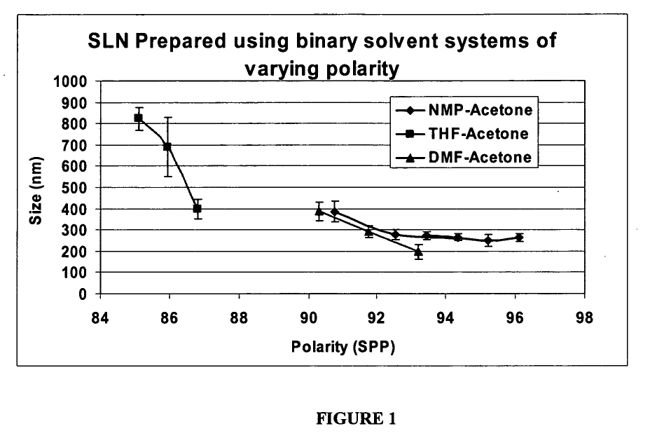

Typically, particle size of the solid lipid nanoparticles of the invention can be a function of polarity of the organic phase. The organic phase is generally a binary solvent system, and the ability to control size allows optimization of SLN uptake and clearance. Particle size depends upon the solvent polarity parameter of the solvent system, which is generally described by the following equation:
SPP=SPP_{A}*V_{A}+SPP_{B}*(1−V_{A})

Wherein SPP is the solvent polarity parameter of the binary solvent system; SPP_{A} is the solvent polarity parameter of component A; SPP_{B} is the solvent polarity parameter of component B; V_{A} is the volume fraction of component A; and (1−V_{A}) is the volume fraction of component B. This relationship is shown further in FIG. 1.

It is understood that the binary solvent system can comprise more than two solvents. In such a case, the solvent polarity parameter of the binary solvent system (SPP) of the overall system would be the sum of the volume fraction weighted solvent polarity parameters for each component in the system.

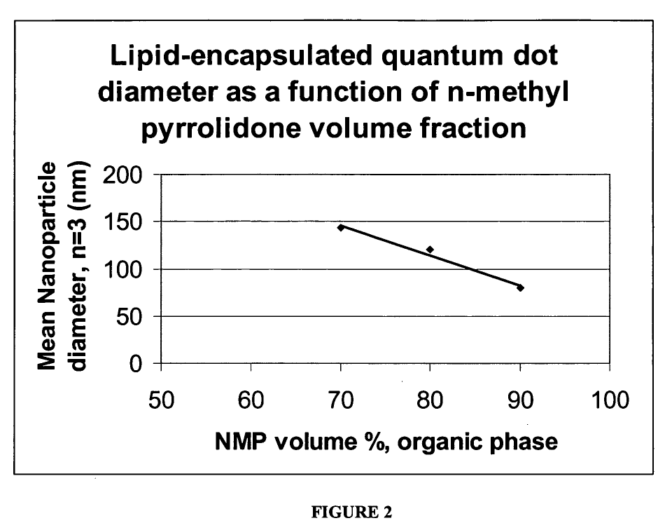

The solid lipid nanoparticles of the invention are size-tunable by the adjustmnent of solvent polarity, as shown in FIG. 2. By adjusting the volume fractions of the organic solvents, miscibility with water is changed which alters the tendency for the aqueous phase to mix with the organic phase, which consequently affects lipid packing as it is exposed to water. The same general size trends are observed for nanoparticles encapsulating a biologically active agent; a pharmaceutically active agent; a magnetically active agent, for example, iron oxide nanoparticles; or an imaging agent, for example, one or more quantum dots. High solvent polarity draws water in more rapidly, producing smaller nanoparticles, whereas a slower influx of water drawn into the organic phase by increasing acetone volume fraction and decreasing the n-methyl pyrrolidone fraction (NMP is highly miscible with water, with acetone being somewhat less miscible) results in slower lipid packing transitions, and thus larger particles.

A solvent system can be selected based on the solvent polarity parameters (SPP) for the component solvents, which determines miscibility in water, which in turn determines how rapidly and readily the aqueous phase is influxed to the organic phase upon phase inversion. SPP can be determined from Snyder's solvent polarity index. Illustrative examples of solvents, with their respective SPP, are acetone (5.4), dimethyl formamide (6.4), tetrahydrofuran (4.2), and toluene (2.3).

Typically, species which are more polar, such as DMF and acetone, as well as NMP (which is miscible with water) tend to rapidly draw in water to the organic phase, whereas somewhat less polar species, such as toluene, typically do not have that tendency to the same extent. The influx of water can have an effect on lipid inward packing to form SLN. Should water move into the organic phase rapidly, smaller particles typically form; if water moves in more slowly (by increasing the volume fraction of a less-polar species in the organic phase), then the lipid packing transition is somewhat slower, resulting in a relatively larger particle, as lipid domains in a given area have time to aggregate together in the packing reaction, entrapping everything within regardless of hydrophobicity/hydrophilicity. Thus, with a binary solvent system comprising toluene and acetone, increasing the toluene fraction relative to acetone in the organic phase can result in larger particles relative to a solvent system wherein the acetone fraction is increased relative to toluene.

The lipids can be “hard fats,” which can be characterized by the ability to become very solid at room temperature; they are strong enough to entrap any species, hydrophilic or hydrophobic, and form a “crust” upon phase inversion which entraps its components for months at a time. The polymeric agents (e.g., the functionalized polymers) can serve as a “crystalline nucleus” which strengthens SLN further by packing lipid domains together and imparting a charge around the neutral lipid, which keeps the SLNs well dispersed without aggregation.

2. Solvent Systems

Because a solvent is used, the present method can obviate the need for melting and solidification steps in the preparation of the SLN. Binary solvents contemplated for use in the present system and methods include, but are not limited to, N-methylpyrolidinone-acetone, tetrahydofuran-acetone, dimethylformamide-acetone, and NMP-acetone-toluene. For example, suitable solvents include combination of N-methylpyrolidinone, tetrahydofuran, dimethylformamide, and toluene. It is understood that other polar organic solvents can be used in connection with the invention, for example, dimethylsulfoxide (DMSO), N-alkyl pyrrolidone(s), and azones. Individual solvents and solvent combinations, and their relative volume fractions, can be selected to provide the solvent polarity parameter (SPP) for the solvent system.

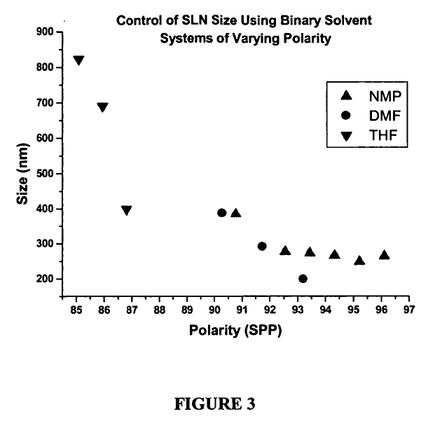

In one aspect, particle size can be modulated by selection of the binary solvent system used in the organic phase. Typically, the higher the solvent polarity parameter of the binary solvent system, the smaller the particle size of the resultant solid lipid nanoparticles. In one aspect, a suitable solvent polarity parameter for the binary solvent system can be from about 80 to about 100, for example, from about 85 to about 100, from about 90 to about 100, from about 90 to about 95, or from about 95 to about 100. The relationship between particle size and solvent polarity parameter is illustrated in FIG. 3.

In the methods of the invention of preparing solid lipid nanoparticles, the lipid is typically dissolved in the binary solvent system of the organic phase before the organic phase is combined with the aqueous phase. It is also understood that various components to be incorporated into the solid lipid nanoparticles of the invention can be introduced by dissolution in the binary solvent system; for example, biologically active agents, pharmaceutically active agents, imaging agents, magnetically active agents, and polyethers can all be dissolved in the binary solvent system.

It is understood that the binary solvent system can comprise more than two solvents. For example, the system can comprise three, four, five, six, seven, eight, nine, or ten solvents. It is also understood that, in a further aspect, a single organic solvent having a suitable polarity can be used as the binary solvent system.

It is also understood that the disclosed binary solvent systems can be used in combination with the compositions of the invention, methods of the invention, products of the invention, and applications of the invention.

3. Aqueous Phase

In the methods of the invention of preparing solid lipid nanoparticles, the functionalized polymers are typically dissolved in the aqueous phase before the organic phase is combined with the aqueous phase. It is also understood that various components to be incorporated into the solid lipid nanoparticles of the invention can be introduced by dissolution in the aqueous phase; for example, biologically active agents, pharmaceutically active agents, imaging agents, magnetically active agents, and polyethers can all be dissolved in the aqueous phase.

It is understood that the disclosed aqueous phases can be used in combination with the compositions of the invention, methods of the invention, products of the invention, and applications of the invention.

4. Phase Inversion

In a system of two immiscible liquids, for example an aqueous phase and an organic phase, there are two general types of dispersions which can be formed depending on the conditions of the system—“water-in-oil” and “oil-in-water.” Typically, a “water-in-oil” dispersion is a dispersion formed when the aqueous phase is dispersed in the organic phase and an “oil-in-water” dispersion is a dispersion which is formed when the organic phase is dispersed in the aqueous phase. Phase inversion is the phenomenon whereby the phases of a liquid-liquid dispersion interchange such that the dispersed phase spontaneously inverts to become the continuous phase and vice versa under conditions determined by the system properties, volume ratio and energy input. Thus, by definition, the phase inversion point is the holdup of the dispersed phase for a system at which this transition occurs. The solid lipid nanoparticles of the invention form spontaneously during phase inversion when the organic phase is combined with the aqueous phase.

5. Layer-by-Layer Assembly

In one aspect, the solid lipid nanoparticles of the invention can be prepared having a single layer of functionalized polymer at the exterior of the particle, or more than one layer of functionalized polymer at the exterior of the particle. In the preparation, each layer can be added to the solid lipid nanoparticles sequentially. That is, in one aspect, a first functionalized polymer can be dissolved in the aqueous phase, which is combined with the organic phase, which contains a lipid. When the organic phase and aqueous phase are combined, the solid lipid nanoparticles of the invention are formed as an aqueous suspension. At least a portion of the first functionalized polymer is at the exterior of the solid lipid nanoparticle, thereby comprising a first layer surrounding the solid lipid nanoparticle.

In a further aspect, further layers can be formed at the exterior of the solid lipid nanoparticles by admixing a second functionalized polymer having at least one ionic or ionizable moiety that is complementary to the ionic or ionizable moiety of the first functionalized polymer. That is, a complementary second functionalized polymer can then be added to the suspension of solid lipid nanoparticles after formation of the solid lipid nanoparticles. In various aspects, the functionalized polymer can be added as a solid, as a liquid, as a solution, as a suspension, or as an emulsion. When added, the second functionalized polymer can form a layer at the exterior of the solid lipid nanoparticle, substantially surrounding the first functionalized polymer, thereby comprising a second layer surrounding the solid lipid nanoparticle.

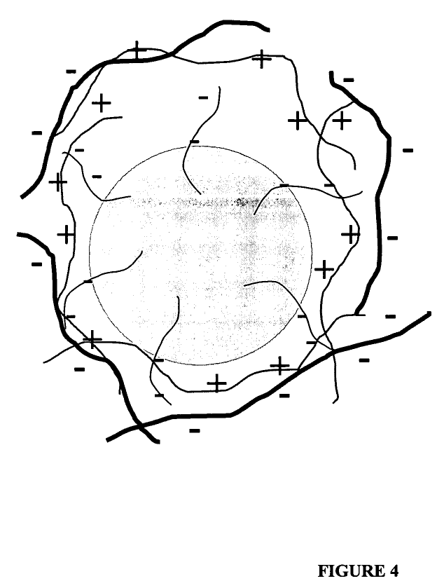

In a further aspect, a complementary third functionalized polymer can then be added to the suspension of solid lipid nanoparticles. In various aspects, the functionalized polymer can be added as a solid, as a liquid, as a solution, as a suspension, or as an emulsion. When added, the third functionalized polymer can form a layer at the exterior of the solid lipid nanoparticles, substantially surrounding the second functionalized polymer, thereby comprising a third layer surrounding the solid lipid nanoparticle. A schematic that illustrates, in one aspect, the layered structure of the solid lipid nanoparticles of the invention is shown in FIG. 4.

It is understood that further complementary functionalized polymers can be then added to form further layers at the exterior of the solid lipid nanoparticles. It is also understood that, in further aspects, more than one functionalized polymer can be added and that the addition can be simultaneous rather than sequential.

6. Inclusion of Surface Active Agents

In one aspect, surface active agents, for example polyethers, biologically active agents, or pharmaceutically active agents, can be included in the solid lipid nanoparticles of the invention during, or subsequent to, preparation of the solid lipid nanoparticles. For example, one or more surface active agents can be dissolved or suspended in the organic phase during preparation of the solid lipid nanoparticles. In a further example, one or more surface active agents can be dissolved or suspended in the aqueous phase during preparation of the solid lipid nanoparticles. In an even further example, one or more surface active agents can be added to the suspension of nanoparticles subsequent to preparation.

In one aspect, when one or more surface active agents are present in the organic phase and/or aqueous phase, the surface active agent(s) can be located at the interior of the solid lipid nanoparticle or (i.e., encapsulated) at the exterior of the solid lipid nanoparticle (i.e., covalently or noncovalently bonded to the functionalized polymer(s) present at the exterior). In a further aspect, when one or more surface active agents are admixed subsequent to formation of the solid lipid nanoparticles, the surface active agent(s) can be located at the exterior of the solid lipid nanoparticle (i.e., covalently or noncovalently bonded to the functionalized polymer(s) present at the exterior).

7. Inclusion of Payloads

In one aspect, payloads, for example biologically active agents, pharmaceutically active agents, magnetically active agents, imaging agents, or a mixture thereof, can be included in the solid lipid nanoparticles of the invention during, preparation of the solid lipid nanoparticles. For example, one or more payloads can be dissolved or suspended in the organic phase during preparation of the solid lipid nanoparticles. In a further example, one or more payloads can be dissolved or suspended in the aqueous phase during preparation of the solid lipid nanoparticles.

In one aspect, when one or more surface active agents are present in the organic phase and/or aqueous phase, the surface active agent(s) can be located at the interior of the solid lipid nanoparticle or (i.e., encapsulated).

In a further aspect, simultaneous encapsulation of both hydrophilic and hydrophobic payloads comprising small molecules and/or proteins can be accomplished with the methods of the invention. In one aspect, a hydrophobic payload, for example a steroid, and a neutral lipid can be dissolved in a binary solvent and a first functionalized polymer, for example poly-L-lysine, and a hydrophilic payload, for example a protein, can be dissolved in an aqueous phase. In this aspect, when the binary solvent system and the aqueous phase are combined, solid lipid nanoparticles are formed, wherein both the hydrophobic steroid and the hydrophilic protein can be encapsulated by the solid lipid nanoparticles.

D. Applications of Solid Lipid Nanoparticles

It is understood that the solid lipid nanoparticles of the invention can be used in connection with any application of lipid nanoparticles known to those of skill in the art. However, it is also understood that, in various specific aspects, the solid lipid nanoparticles of the invention can be used in connection with, for example, tumor targeting therapeutic systems, thermoresponsive payload delivery, functionalized quantum dots, magnetic-driven targeting, multimodal diagnostic therapeutic systems, trans-blood-brain-barrier delivery, trans-lipid-bilayer delivery, subcellular organelle targeting, cosmetic formulations, and ink formulations.

1. Tumor Targeting Therapeutic Systems

In one aspect, the solid lipid nanoparticles of the invention can be used in connection with a tumor targeting therapeutic system comprising a solid lipid nanoparticle having a biologically active agent at the exterior of the solid lipid nanoparticle, and a pharmaceutically active agent encapsulated within the solid lipid nanoparticle; wherein the biologically active agent comprises at least one enzyme, antibody, targeting protein, or signal protein. In one aspect, the enzyme can be a targeting enzyme.

Typically, the enzyme or enzymes located at the exterior of the solid lipid nanoparticle are selected to be specific for the tissue of a tumor. Consequently, the solid lipid nanoparticle is directed to the site of the tumor by the specificity of the enzyme or enzymes. In a further aspect, the enzyme or enzymes can act to “digest” the outer capsule of a solid tumor, so that the particle can diffuse more rapidly.

In one aspect, the targeting enzyme can be a matrix metalloproteinase. The extracellular matrix (ECM) of tumors is typically a tough dense fibrous barrier to pharmaceutic transport. A targeting enzyme, in concert with a pharmaceutically active agent payload, can penetrate through this dense tissue to deliver the agent to the tumor core. The result can be more homogenous drug levels intratumorally with higher efficacy potentially because therapeutic levels are, therefore, higher and the pharmaceutically active agent is no longer constrained to the periphery of a tumor.

Matrix metalloproteinases (MMPs) are well-known to those of skill in the art and are a family of zinc metallo-endopeptidases secreted by cells, and are responsible for much of the turnover of matrix components. They are included in the “MB clan” of metallopeptidases, generically referred to as “Metzincins,” containing the motif HEXXHXXGXXH as the zinc binding active site. MMPs are involved in a wide range of biological processes, including tissue remodeling and also modification or release of biological factors. Pathological processes involving MMPs include tumor growth and migration, fibrosis, arthritis, glaucoma, lupus, scleroderma, cirrhosis, multiple sclerosis, aortic aneurysms, infertility, and many more diseases. Proteinase inhibitors such as α1-proteinase inhibitor, antithrombin-III and α2-macroglobulin are selectively cleaved by MMPs. Growth factors such as IL-1α and pro-TNF-α are cleaved by MMPs, as are IGF binding protein-3 and IGFBP-5.

In one aspect, the pharmaceutically active agent is an anti-tumor treatment. The anti-tumor treatment can be a chemotherapeutic, for example, an antineoplastic drug.

In a further aspect, the solid lipid nanoparticle can be functionalized with a surface active agent, for example, an antibody. In this aspect, the solid lipid nanoparticle can also be functionalized with one or more payloads, for example, a chemotherapeutic agent and/or s radiosensitizer, and/or a MR imaging agent (e.g., Gadolinium and Magnetite), and/or an agent that enables localization (antibody and/or magnetite) and/or imaging agent such as a quantum dot, and/or enzymes that can promote diffusion of the solid lipid nanoparticle into solid tumors, and /or agents that can inhibit angiogenesis.

By combining the tumor targeting function of the biologically active enzymes with the anti-cancer therapeutic function of the pharmaceutically active chemotherapeutic, the solid lipid nanoparticle of the invention tumor targeting therapeutic systems can provide superior treatment when administered to a subject. In a specific aspect, the solid lipid nanoparticles of the invention can be used in connection with a tumor targeting therapeutic system comprising a solid lipid nanoparticle having a biologically active agent at the exterior of the solid lipid nanoparticle, and a pharmaceutically active agent encapsulated within the solid lipid nanoparticle; wherein the biologically active agent comprises at least one enzyme, antibody, targeting protein, or signal protein and wherein the pharmaceutically active agent comprises the combination of a radiosensitizer and a chemotherapeutic.

In a further aspect, the tumor targeting therapeutic system can further comprise an imaging agent.

2. Thermoresponsive Payload Delivery

In one aspect, the solid lipid nanoparticles of the invention can be used in connection with a thermoresponsive payload delivery system comprising a first solid lipid nanoparticle, wherein the first solid lipid nanoparticle has a first payload and a first melting temperature and, optionally, a second solid lipid nanoparticle, wherein the second solid lipid nanoparticle has a second payload and a second melting temperature, and wherein the second melting temperature is higher than the first melting temperature.

It is understood that the melting temperature of the solid lipid nanoparticles of the thermoresponsive payload delivery systems can be modulated by selecting a first lipid having a first melting point of a first temperature and combining the first lipid with a second lipid having a second melting point of a second temperature, as disclosed herein.

Thermoresponsive payload delivery system can also be referred to as thermosensitive systems.

In one aspect, the thermoresponsive payload delivery system can also include a pharmaceutically active agent or a magnetically active agent.

It is understood that, in one aspect, the disclosed thermoresponsive payload delivery systems can be used in a method of thermoresponsive payload delivery within a subject comprising the steps of administering an effective amount of the thermoresponsive payload delivery system to a subject; and applying heat to a location within the subject, thereby increasing the temperature of the location above the first melting temperature and melting the solid lipid nanoparticle, whereby the first payload is delivered to the location within the subject.

It is also understood that, in a further aspect, the disclosed thermoresponsive payload delivery systems can be used in a method of thermoresponsive payload delivery within a subject comprising the steps of administering an effective amount of the thermoresponsive payload delivery system to a subject; applying a first heat to a first location within the subject, thereby increasing the temperature of the first location above the first melting temperature and melting the first solid lipid nanoparticle, whereby the first payload is delivered to the first location within the subject; and applying a second heat to a second location within the subject, thereby increasing the temperature of the second location above the second melting temperature and melting the second solid lipid nanoparticle, whereby the second payload is delivered to the second location within the subject. In a further aspect, the first location and the second location can be different.

By combining the controlled release function of the modulated melting temperature thermoresponsive payload delivery systems with the therapeutic function of a pharmaceutically active agent, the solid lipid nanoparticle of the invention thermoresponsive payload delivery systems can provide superior treatment when administered to a subject.

In one aspect, bovine serum albumin (BSA), quantum dots, and iron oxide can be encapsulated within the solid lipid nanoparticles with the disclosed lipid entrapment strategy, using the lipids SOFTISAN® 100, SOFTISAN® 142, and SOFTISAN® 154. These three lipids can be used separately in the organic phase, or alternatively, blended together at certain ratios. In one aspect, the choice of lipid(s) can modulate the controlled release properties of the solid lipid vesicles. The lipids of the invention are typically characterized by narrow ranges of melting points, within which complete release of contents can be achieved. While the use of SOFTISAN® 100, for example, has a melting point of from about 33° C. to about 35° C., by blending with an equal fraction of SOFTISAN® 142, the melting point can be raised considerably to a point from about 34° C. to about 38° C. Likewise, blending of SOFTISAN® 142 and 154 formulations can produce melting points in between the two individual melting points. By developing a lipid-encapsulated drug delivery system which is thermoresponsive within narrow ranges, several therapeutic avenues are possible.

In one aspect, an application for this technology is radiofrequency ablation, which relies on current-generated heat to debulk tumors. This procedure is FDA-approved for the treatment of liver cancers. The therapeutic temperature range of the technique typically ranges from 43° C. to about 100° C. Lipid melting point temperatures of SOFTISAN® 142 and 154 fall within this range, and the solid lipid nanoparticles can be engineered to bear protein cargoes and tumor antibodies. Thus, lipid nanoparticles could be used for thermosensitive, site-specific drug delivery upon hyperthermia induced by RF ablation. Additionally, the drug-loaded nanoparticles can be delivered through the ablation catheter itself during the procedure. This can be a particularly useful application in the field of ablation, since the liver's rich blood supply, a “heat sink,” can often limit the effectiveness of ablation sessions, necessitating coincident chemotherapy in many cases to prevent tumor recurrence at ablation boundaries. A further, simpler application of thermosensitive lipid carriers can be locally applied hyperthermia at the site of interest to promote drug release. This can be especially applicable to tumors closer to the skin's surface, such as neck cancers which involve tumors present in multiple lymph nodes.

Thermosensitive lipid carriers need not carry only drugs to be effective in therapy. Magnetically active agents—such as, for example, iron oxide nanoparticles, which have also been shown to be powerful inductive heating agents for ablation therapy—can also be encapsulated. The iron oxide can act as a resistive element to current, and can then transform into a tumor killing element that is injected directly into tumors. Furthermore, tumor cells have been shown to be more sensitive to 41° C.+ heat than normal cells, making lipid-encapsulated magnetic nanoparticle-based radiofrequency ablation a promising therapeutic avenue (See Berry, C. C., and A. S. G. Curtis, 2003, Functionalization of magnetic nanoparticles for applications in biomedicine, J Phys D Appl Phys 36(13):R198-R206; Hilger, I., K. Fruhauf, W. Andra, R. Hiergeist, R. Hergt, and W. A. Kaiser, 2002, Heating potential of iron oxides for therapeutic purposes in interventional radiology, Acad Radiol 9(2):198-202; Hilger, I., R. Hergt, and W. A. Kaiser, 2000, Effects of magnetic thermoablation in muscle tissue using iron oxide particles—An in vitro study, Invest Radiol 35(3):170-179).

The lipid encapsulation strategy of the present invention provides a powerful tool for the design of drug delivery and imaging tools for in vitro and in vivo applications. Solid lipid nanoparticles are size-tunable; amenable to multiple surface functionalities such as peptides, DNA, proteins, and polymers; can be produced rapidly in mass quantities using GRAS (Generally Recognized As Safe) components in a single-step without surfactants or cooling/melting steps; and can bear multiple cargos, for example surface functional agents or payloads (e.g., quantum dot nanocrystals, iron oxide nanoparticles, and proteins). Lipid carriers can be functionalized with poly(styrene-4-sodium sulfonate) to rapidly traverse the plasma membrane of cells to target intracellular organelles, or can be electrostatically-coated with a poly-L-lysine coating to conjugate proteins or oligonucleotides to the solid lipid nanoparticle surface for flow cytometric sorting of DNA sequences or viruses, or for in vivo targeting applications such as cancer imaging and drug delivery.

In a further aspect, the thermoresponsive payload delivery system can be functionalized to release the payload upon laser ablation. In an even further aspect, the thermoresponsive payload delivery system can be a multimodal system, wherein the solid lipid nanoparticle can contain a payload, for example gold nanoparticles, that that can increase in temperature in response to an RF energy source and thereby melt the lipid component of the solid lipid nanoparticle and release the payload.

3. Functionalized Quantum Dots

In one aspect, the solid lipid nanoparticles of the invention can be used in connection with a functionalized quantum dot. For example, a functionalized quantum dot can be one or more quantum dots encapsulated within the solid lipid nanoparticles of the invention.

In a further aspect, in the functionalized quantum dot of the invention, the first functionalized layer can further comprise at least one cysteine-rich protein, at least one metallothionein-rich protein, or a mixture thereof. In a further aspect, in the functionalized quantum dot of the invention, the quantum dot can further comprise a second functionalized polymer, wherein the second functionalized layer further comprises at least one cysteine-rich protein, at least one metallothionein-rich protein, or a mixture thereof. In a further aspect, in the functionalized quantum dot of the invention, the quantum dot can further comprise a third functionalized polymer, wherein the third functionalized layer further comprises at least one cysteine-rich protein, at least one metallothionein-rich protein, or a mixture thereof.

Quantum dots (QDs), or semiconducting nanocrystals, which, due to their unique optical properties, are used as cellular and tissue imaging agents. In the synthesis of QD, a nanometer-sized crystal (usually CdSe) is capped with a larger bandgap, secondary layer of ZnS for enhanced optical behavior. The absorption of a photon of light by the semiconducting material and subsequent emission of a lower energy photon results in fluorescence. However, applications of QD for in vivo imaging have been limited due to concerns over potential cytotoxicity caused by nanocrystal core heavy metal release due to shell dissociation. See Derfu, A. M., et al., Nano Letters 2004, 4 (1) 11-18.

These heavy metals can include Cd for emission in the visible light range, and Pb for infrared imaging. An additional challenge posed by in vivo QD utilization is the attainment of enhanced circulation and tissue half-life. The QD can serve as an intact biomarker at its specific site for as long a time as is desired; thus, the structure can be resistant to undesirable immune responses, such as phagocytotic uptake, and other mechanisms leading to degradation of the nanocrystal.

Quantum dots have highly desirable optical properties which can make them suitable candidates for biological imaging, such as high quantum efficiency, size-tunable emission wavelengths, small nanoscale feature sizes, and the capacity for all nanocrystals to be excited by one excitation wavelength, which obviates the need for multiple illumination sources. Quantum dots have been applied to in vivo imaging of cancer and tissues, and has extensive use currently in biological imaging applications in which a biofunctional ligand is attached to the nanocrystal for specific targeting of proteins (See Akerman, M. E., W. C. W. Chan, P. Laakkonen, S. N. Bhatia, and E. Ruoslahti, 2002, Nanocrystal targeting in vivo, Proceedings of the National Academy of Sciences of the United States of America 99(20):12617-12621); Ballou, B., B. C. Lagerholm, L. A. Ernst, M. P. Bruchez, and A. S. Waggoner, 2004, Noninvasive imaging of quantum dots in mice, Bioconjug Chem 15(1):79-86; Gao, X. H., Y. Y. Cui, R. M. Levenson, L. W. K. Chung, and S. M. Nie, 2004, In vivo cancer targeting and imaging with semiconductor quantum dots, Nature Biotechnology 22(8):969-976; Larson, D. R., W. R. Zipfel, R. M. Williams, S. W. Clark, M. P. Bruchez, F. W. Wise, and W. W. Webb, 2003, Water-soluble quantum dots for multiphoton fluorescence imaging in vivo, Science 300(5624):1434-1436; Lidke, D. S., P. Nagy, R. Heintzmann, D. J. Arndt-Jovin, J. N. Post, H. E. Grecco, E. A. Jares-Erijman, and T. M. Jovin, 2004, Quantum dot ligands provide new insights into erbB/HER receptor-mediated signal transduction, Nature Biotechnology 22(2):198-203). In order to prepare quantum dots for biomedical applications, researchers have surface modified quantum dots with triblock copolymers, phospholipid micelles, and cholesterol-bearing pullulan (See Dubertret, B., P. Skourides, D. J. Norris, V. Noireaux, A. H. Brivanlou, and A. Libchaber, 2002, In vivo imaging of quantum dots encapsulated in phospholipid micelles, Science 298(5599):1759-1762; Gao, X. H., Y. Y. Cui, R. M. Levenson, L. W. K. Chung, and S. M. Nie, 2004, In vivo cancer targeting and imaging with semiconductor quantum dots. Nature Biotechnology 22(8):969-976; Hasegawa, U., S. I. M. Nomura, S. C. Kaul, T. Hirano, and K. Akiyoshi, 2005, Nanogel-quantum dot hybrid nanoparticles for live cell imaging, Biochem Bioph Res Co 331(4):917-921). These techniques are often time-consuming, and involve chemical modification of the quantum dot surface with the effects on quantum efficiency being unknown. Furthermore, these techniques provide protection to quantum dots from uptake and degradation mechanisms (e.g., surface oxidation), but do not facilitate their entry into cells, which is highly desired in biology and medicine. The solid lipid nanoparticle encapsulation strategy of the invention used for paramagnetic nanoparticle entrapment can also be applied to the development of a vehicle for transporting quantum dot nanocrystals. The lipophilic nanoparticles enable intracellular transport without energy-dependent endocytosis.

In vitro studies have demonstrated that QD do not affect cell viability in a hepatocyte model, as well in a metastatic tumor cell mode. See Voura, E. B., et al., Nature Medicine 2004, 10(9) 993-998. In both of these cases, cells were introduced inside the cell for labeling purposes. Microinjection and/or endocytosis are two mechanisms by which this can be achieved. Once inside the cell, QD do not typically traverse extracellularly, and can even remain within daughter generations. See Watson, A., et al., Biotechniques 2003, 34:296-30.

However, this mechanism of individual, intracellular labeling cannot be realistically achieved in vivo, and in addition, many nanobioconjugates rely on cell surface markers, such as the antibody and peptide-conjugated QD described above. In the body, such QD conjugates aimed at external markers are susceptible to uptake by the phagocytotic cells of the reticuloendothelial system (RES), which includes the liver, spleen, and kidney, in which case they may be exposed to harsh, oxidative environments. Surface engineering designs have included encapsulation in phospholipids micelles, covalent immobilization to block co-polymer and poly(p-phenylene vinylene) backbones, surface functionalization via organic phosphines and proteins, and high molecular weight PEGylation. See Dubertret, B., et al., Science 2002, 298: 1759-1762. Skaff, H., et al., J. Am. Chem. Soc. 2004, 126, 11322-11325; Kim, S., et al., Nature Biotech. 2004, 22(1), 93-97; Ballou, B., et al., Bioconjugate Chem. 2004, 15, 79-86. No conventional approach eliminates surface oxidation completely.

One aspect of the compositions of the invention and methods is the reduced toxicity of encapsulated quantum dots. This aspect is accomplished through surface modification of the quantum dot. One of the essential components of this approach is prolongation of in vivo half-life and therefore reduction in bio-degradation. The surface coating of the present invention results in increased stability of the quantum dots, resulting in a lower dose and an increased likelihood that they will be cleared prior to degradation. Additionally, the compositions of the invention and methods sequester the heavy metal degradation products by surface engineering of the quantum dot, which can help limit long-term toxicity.

A further aspect of the compositions of the invention and methods is a novel QD based on the encapsulation the QD within a lipid coating, the surface of which has been engineered to bear cysteine-rich peptide segments. In this encapsulation paradigm, the lipid coat can serve to confine the quantum effect (by preserving the core-shell), as well as to contain possible free Cd within the particle environment, thus addressing both longevity and toxicity aspects essential for in vivo QD applications. The disclosed functionalized quantum dot can include features to present a variety of information such as charges and large, impermeable molecules, in a single synthesis step.

The development of a probe which is internalized by cells by an energy-independent mechanism has broad consequences in the application of nanotechnology to biology and medicine. Applications of this technology include the labeling of organelles such as mitochondria in living cells, the targeting of specific tissues in vivo, and ADME studies on therapeutics. Given the demonstrated capacity to encapsulate iron oxide nanoparticles, it is also possible to develop magnetooptical probes which take advantage of the contrast enhancing and magnetic guidance properties of CLIO-NP for imaging and drug delivery, as well as the unique, powerful optical properties of quantum dots.

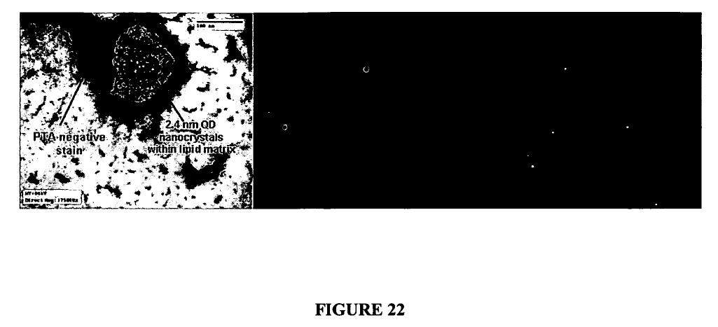

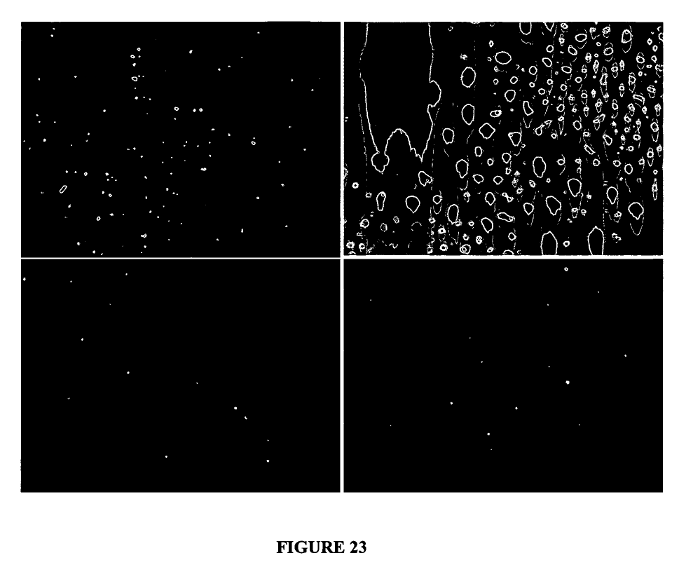

TEM analysis of SLN-QD and fluorescence microscopic analysis of SLN-QD are shown at FIG. 22 and FIG. 23, respectively.

4. Quantum Confinement and Containment

While in vitro cellular assays are useful in assessing trends in cytocompatibility, they provide limited information with respect to biodistribution and clearance. The body's natural clearance and processing mechanisms for particulates, i.e., phagocytosis and glomerular filtration, typically cannot be reproduced or simulated in an in vitro system. Therefore, data related to tissue toxicity are obtained from studies with animal subjects. The effects on a tissue level must be extensively investigated in order for quantum dot (QD) clinical applications to become a reality. Ballou et al., have demonstrated that by coating a QD with polyethylene glycol (PEG), a molecule that can confer RES evading properties to nanoparticles in circulation; QD-tissue interactions can be substantially reduced. Ballou, B., et al., Noninvasive imaging of quantum dots in mice, Bioconjugate Chemistry, 2004, 15(1): p. 79-86. This observation is consistent with the fact that cadmium has a half-life in the body of up to 30 years, and normally accumulated in the proximal tubule cells of the kidney and can be retained in humans throughout life. Jones, S. G., et al., Intracellular Cadmium Mobilization Sequelae, Toxicology, 1990, 61(1): p. 73-83. This accumulation can be due to the inability of the tubule cells to secrete the cadmium. Divalent cadmium enters the kidney bound to metallothionein, a metal chelating protein synthesized by the liver. Once in the proximal tubule cell, the Cd-metallothionein complex is broken down in the lysosome to yield free cadmium, which stimulates endogenous proximal tubule cell production of metallothionein resulting in the recycling of the metal. This process continues until excess cadmium entry into the cell causes cell death. Nordberg, M., Studies on Metallothionein and Cadmium, Environmental Research, 1978, 15(3): p. 381-404; Nordberg, M. and G. F. Nordberg, Toxicological aspects of Metallothionein, Cellular and Molecular Biology, 2000, 46(2): p. 451-463; Jin, T. Y., J. Lu, and M. Nordberg, Toxicokinetics and biochemistry of cadmium with special emphasis on the role of metallothionein, Neurotoxicology, 1998, 19(4-5): p. 529-535.

Since oxidation of the surface can be a primary contributor to core-shell breakdown and reduction in QD fluorescence lifetime, much research has been focused on encapsulation of QD in micron-sized moieties such as lipid and polymeric vesicles. Recently, Nie et al., have demonstrated that concentrations in the order of 106 QD/cell can be achieved without significantly affecting cell viability by encapsulating of QD polymer-derived vesicles. Watson, A., X. Y. Wu, and M. Bruchez, Lighting up cells with quantum dots, Biotechniques, 2003, 34(2): p. 296-+. Other strategies that have been employed to improve tissue specific or cellular localization of QD include modification of QD nanocrystal surfaces through bioconjugation chemistry to bear antibodies (Gao, X. H., et al., In vivo cancer targeting and imaging with semiconductor quantum dots. Nature Biotechnology, 2004, 22(8): p. 969-976.), peptides (Akerman, M. E., et al., Nanocrystal targeting in vivo, Proceedings of the National Academy of Sciences of the United States of America, 2002, 99(20): p. 12617-12621.), and receptor ligands (Rosenthal, S. J., et al., Targeting cell surface receptors with ligand-conjugated nanocrystals, Journal of the American Chemical Society, 2002, 124(17): p. 4586-4594.), as well as encapsulation in phospholipid micelles (Dubertret, B., et al., In vivo imaging of quantum dots encapsulated in phospholipid micelles, Science, 2002, 298(5599): p. 1759-1762.). Using these strategies, QD have been explored in clinical research applications ranging from tumor imaging (Akerman, M. E., et al., Nanocrystal targeting in vivo, Proceedings of the National Academy of Sciences of the United States of America, 2002, 99(20): p. 12617-12621.), sentinel lymph node mapping (Kim, S., et al., Near-infrared fluorescent type II quantum dots for sentinel lymph node mapping, Nature Biotechnology, 2004, 22(1): p. 93-97.), concentration-dependent biosensing of a specific substance (Medintz, I. L., et al., Self-assembled nanoscale biosensors based on quantum dot FRET donors, Nature Materials, 2003, 2(9): p. 630-638; Medintz, I. L., et al., A fluorescence resonance energy transfer-derived structure of a quantum dot-protein bioconjugate nanoassembly, Proceedings of the National Academy of Sciences of the United States of America, 2004, 101(26): p. 9612-9617.), and tumor metastasis tracking (Voura, E. B., et al., Tracking metastatic tumor cell extravasation with quantum dot nanocrystals and fluorescence emission-scanning microscopy, Nature Medicine, 2004, 10(9): p. 993-998.). These results are very promising with respect to utilization of QD as diagnostic markers for screening of various pathologies. Other strategies as discussed earlier have focused on increasing circulation times of QD by surface functionalization of the QD nanocrystals using PEG (Ballou, B., et al., Noninvasive imaging of quantum dots in mice, Bioconjugate Chemistry, 2004, 15(1): p. 79-86.).

One strategy to address the toxicity issues related to free cadmium release from QD is use of technologies that allow for protection of the QD in the biological environment from oxidative processes. Using such a strategy, QD with improved resistance to oxidation and diminished cytotoxicity can be developed via the application of a paradigm referred to as quantum confinement and containment (QCC). The key principle of this strategy is the manipulation of the environment around the quantum dot so as to diminish its accessibility to oxidative species, while providing a means of sequestering any free cadmium to the QD environment, thus diminishing Cd-associated toxicity. This strategy differs in a significant manner from current approaches as it involves encapsulation of the QD in a solid nano-scale matrix, for example a solid lipid nanoparticle, as opposed to derivatization of the QD structure through bioconjugation chemistries. The primary advantages of using a nano-scale matrix over a micron size carrier is that the nano-particulate matter can enter the capillary bed and is easily taken by cells via receptor mediated process. Furthermore, additional significant advantages of this approach include a higher localized dose, as the QD is delivered as packet of information as opposed to individual QD moieties, and the ease of engineering surface information to achieve optimized cell-specific or tissue specific targeting. The latter enables the use of a high throughput approaches to optimize antioxidant encapsulation environments and Cd sequestering.

QCC can be achieved around a QD by encapsulating the QD in a lipid environment that exhibits high oxidative resistance and biocompatibility. Furthermore, to increase circulation times and achieve sequestering of any free cadmium, the lipid surface and matrix can be functionalized with a peptide moiety that is rich in cysteine residues and further functionalized with high molecular weight poly(ethylene glycol) (PEG), a neutral water soluble polymer that is know to reduce RES uptake and increase clearance of nanoparticulate entities (Woodle, M. C., et al., Prolonged Systemic Delivery of Peptide Drugs by Long-Circulating Liposomes—Illustration with Vasopressin in the Brattleboro Rat, Pharmaceutical Research, 1992, 9(2): p. 260-265; Woodle, M. C. and D. D. Lasic, Sterically Stabilized Liposomes, Biochimica Et Biophysica Acta, 1992, 1113(2): p. 171-199; Woodle, M. C., et al., Versatility in Lipid Compositions Showing Prolonged Circulation with Sterically Stabilized Liposomes. Biochimica Et Biophysica Acta, 1992. 1105(2): p. 193-200.).

Single solid lipid nanoparticles (SLN) derived from a triglyceride lipid SOFTISAN®, which has seen much use in the dermatological arena, were synthesized. Some of the unique properties of SOFTISAN® include high oxidative stability and a melting transition that is very close to physiological temperatures. The process allows for the preparation of functionalized SLN without use of surfactant or heat thereby making it amenable to encapsulation of biological moieties such as proteins, peptides, oligonucleotides and other heat sensitive compounds. Furthermore, various substances have been encapsulated within the functionalized SLN coating including heavy metal MR contrast agents contrast agents such as Gadolinium-DTPA (Ga-DTPA), fluorescently labeled large proteins, such as albumin and hydrophobic drugs such as coumarin-6. By varying the polarity of the lipid solution, SLN can be produced and functionalized in a single step by the addition of water phase containing polyelectrolytes and/or neutral water soluble polymers such as PEG and pluronics. In this system, the stabilization of the SLN can be achieved by the enrichment of the SLN surface with a ionized or ionizable or water-soluble polymer or polymers, which serve to electrostatically or sterically stabilize the SLN colloidal suspension. Using this process, SLN bearing a variety of surface functionality ranging from heparin, poly(acrylic acid), a mucco-adhesive polymer; poly(lysine-HCl), and PEG have been prepared. The presence of surface functionality has been verified by measuring the zeta potential of the particle as a function of pH. These analyses have shown that the isoelectric point of the SLN surface corresponds well with the pKa of the ionizable group in the functional moiety. Several studies have shown size to be a parameter in localization and cellular uptake of SLN (Pang, S. W., et al., Effects of charge density and particle size of poly(styrene/(dimethylamino)ethyl methacrylate) nanoparticle for gene delivery in 293 cells, Colloids and Surfaces B-Biointerfaces, 2002, 26(3): p. 213-222.). The use of a lipid solution with a tunable polarity allows for a significant degree of control over SLN size. SLN ranging in size from about 10 nm to about 1000 nm, for example from about 200 to about 800 nm, can be prepared using this approach. The developed process is amenable to encapsulation of small and large molecules alike. The encapsulation of hydrophilic molecules and hydrophobic small molecules such as coumarin-6 and large proteins such as bovine serum albumin (BSA) occurs without any interference to the surface functionalization. Additionally, the internalization of SLN containing Ga-DTPA, coumarin-6, and BSA within bovine aortic endothelial cells (BAEC) showed that these functionalized SLN do not affect cell viability and are stable in the cellular environment for extended periods of time (range of weeks). Since the lipid environment is not opaque, it is amenable to fluorescence spectroscopy, an important requirement for using QD as an imaging tool. Furthermore, functionalized SLN containing heavy metals such as Gadolinium exhibit good solution stability, i.e., no aggregation was observed in aqueous environments over extended periods of over 3-4 months. We have also shown that by selection of surface functionality, transport of SLN across biological barriers such as the blood brain barrier is attainable. This is of particular significance in enabling the use of QD for CNS imaging applications where tight biological barriers can present challenges.

QD encapsulated in SLN derived from SOFTISAN® (Sasol Gmbh) can be prepared as per the single step process disclosed herein. The size and surface characteristics can be determined using dynamic laser light scattering and zeta potential measurements. In brief, QD (Evident Technologies) will be introduced into the organic phase containing the lipid, and encapsulation of the QD in a SLN environment can be achieved by a phase inversion process, by the addition of a water phase containing the selected surface functionality and a cysteine-rich protein or a metallothionein-rich moiety.

The QD-SLN can then be evaluated for the long-term solution stability (e.g., aggregation and SLN integrity and premature QD release from the lipid matrix). The QD-SLN can then be subjected to aqueous environments that simulated the oxidative environments found in lysosomes and peroxisomes. A step-wise addition process can be performed in order to determine key enzymatic degradation schemes which are detrimental to QD-SLN and commercially-available QD structures. Furthermore, QD-SLN and QD can then be exposed to various pH levels. Using methods by Derfus et al. (Derfus, A. M., W. C. W. Chan, and S. N. Bhatia, Probing the cytotoxicity of semiconductor quantum dots, Nano Letters, 2004, 4(1): p. 11-18.), structural integrity can be disturbed by using high intensity UV light (which can dissolve semiconductor particulates, exposing cadmium), and also air exposure. Following destructive exposure (or no exposure) at various intervals, any released cadmium ion in the medium can be measured to confirm sequestration through complexation to cysteine-rich peptides. The solutions can be assayed for free Cadmium using a Fura-2 colorimetric assay (qualitative) (Hinkle, P. M., E. D. Shanshala, and E. J. Nelson, Measurement of Intracellular Cadmium with Fluorescent Dyes—Further Evidence for the Role of Calcium Channels in Cadmium Uptake (Vol 267, Pg 25553, 1992), Journal of Biological Chemistry, 1993, 268(8): p. 6064-6064.) and atomic absorption (Munoz, J., et al., Development of a method for the determination of inorganic cadmium and cadmium metallothioneins in fish liver by continuous preconcentration on fullerene and flame atomic absorption spectrometry, Journal of Analytical Atomic Spectrometry, 2002, 17(7): p. 716-720.) or inductively coupled plasma mass spectrometry (ICP-OES) (Derfus, A. M., W. C. W. Chan, and S. N. Bhatia, Probing the cytotoxicity of semiconductor quantum dots, Nano Letters, 2004, 4(1): p. 11-18.) (quantitative) methods as published.

In vitro cellular uptake studies can be carried using two important cell types: splenic cells and proximal tubule epithelial cells. Splenic macrophages of the marginal zone have shown high phagocytic activity towards QD in preliminary studies, and proximal tubule epithelial cells are the sites implicated in cadmium trapping, and thus these cells will be the focus of this phase of the project. Splenic macrophages can be isolated and cultured as described in published methods (Deng, J. P., et al., Adrenergic modulation of splenic macrophage cytokine release in polymicrobial sepsis, American Journal of Physiology—Cell Physiology, 2004, 287(3): p. C730-C736.). Rat proximal tubule epithelial cells (RPTEC) can be purchased from and cultured according to instructions provided by Cambrex Bioproducts.

Sub-confluent monolayer cultures of these cells can be exposed to commercial (virgin) QD suspensions, commercial QD suspensions exposed to enzymatic and UV degradation environments, and QD-SLN and QD-SLN exposed to enzymatic and UV degradation environments. The fate of the QD as they undergo cellular uptake can be followed for a two week period using time-lapse fluorescent microscopy (for events such as endosome-lysosome fusion), and the rate and extent of uptake will be quantified. Nunc Lab-Tek II chambered coverglasses will serve as the cell attachment template, as their low thickness and high optical quality make it a suitable platform for high magnification microscopy. If possible, the accumulation of free cadmium in subcellular compartments such as the endosome and lysosome will be followed using Fura-2, a fluorescent marker with high affinity for cadmium as described previously (Hinkle, P. M., E. D. Shanshala, and E. J. Nelson, Measurement of Intracellular Cadmium with Fluorescent Dyes—Further Evidence for the Role of Calcium Channels in Cadmium Uptake (Vol 267, Pg 25553, 1992), Journal of Biological Chemistry, 1993, 268(8): p. 6064-6064.). Image Pro Plus (Media Cybernetics) image analysis software can be used to correlate fluorescence intensities with Cd concentration. Changes in metabolic activity and viability of the cells as function of QD dose and time can also be ascertained. Specifically, any changes in the mitotic activity of the cells and programmed cell death activity using MTT and TUNEL assays, respectively, can be determined by using reagents and protocols from Molecular Probes (Haugland, R. P., Handbook of Fluorescent Probes and Research Products, 9th ed. 2002, 650-651, 612.).

Rats can be injected with solutions used in cellular uptake studies, and the accumulation of QD in various tissue compartments (spleen, liver, proximal tubules) can be followed. Using fluorescence microscopy, tissue sections can be examined under fluorescence and laser-scanning confocal microscopy at various time points after dosing, to establish accumulation patterns in various RES compartments and the lymphatic system. Furthermore, kidney, spleen and liver can be harvested at autopsy and cadmium accumulation will be quantified using flame ionization of plasma enhanced atomic absorption spectroscopy. In addition to these valuable pieces of information, these preliminary studies in rat allows establishment of the benefits of delivery of QD in solid nanoscale carriers, for example the solid lipid nanoparticles of the invention, and their role in diminishing exposure to oxidative environments and mitigating the negative effects associated with free cadmium generation in vivo. The urine of the rat subjects can be monitored on a daily basis for calciuria and proteinuria which have been identified by Nordberg and co-workers as an early marker of cadmium toxicity (Leffler, P. E., T. Y. Jin, and G. F. Nordberg, Differential calcium transport disturbances in renal membrane vesicles after cadmium-metallothionein injection in rats, Toxicology, 2000, 143(3): p. 227-234.). The understanding gained from these studies can be used to design modifications and cadmium sequestering strategies aimed at achieving in vivo lifetimes of QD on the scale of few years and complete renal clearance of free cadmium, respectively.

5. Magnetic-Driven Targeting

In one aspect, the methods of the invention can be a method of delivering at least one biologically active agent, pharmaceutically active agent, magnetically active agent, imaging agent, or a mixture thereof to a location within a subject comprising the steps of administering an effective amount of a solid lipid nanoparticle of the invention encapsulating a magnetically active agent and optionally encapsulating at least one biologically active agent, pharmaceutically active agent, imaging agent, or a mixture thereof, to a subject, and applying a magnetic field to the location, whereby the at least one biologically active agent, pharmaceutically active agent, magnetically active agent, imaging agent, or mixture thereof is delivered to the location.

It is known that magnetically active agents can be influenced by a suitably strong magnetic field. The solid lipid nanoparticles of the invention can comprise a magnetically active agent as a payload. In this aspect, when administered to a subject, the solid lipid nanoparticle can be directed to a location within the subject wherein a magnetic field has been applied to that location of the subject. For example, a specific organ within a subject can be targeted for delivery of the solid lipid nanoparticle having a magnetically active payload by applying a magnetic field proximate to that organ. When combined with other disclosed compositions and methods, this approach can provide a flexible and powerful method of targeted delivery and/or imaging.

In one example, a solid lipid nanoparticle comprising a magnetically active agent, a pharmaceutically active agent, and an imaging agent can be targeted for delivery to a specific location within a subject. That is, after administration to the subject, the solid lipid nanoparticle can be directed to the location within the subject by applying a magnetic field, thereby also delivering the pharmaceutically active agent and the imaging agent to this location.

It is also understood that the magnetically-driven targeting methods can be used in combination with other methods, for example the thermoresponsive payload delivery methods, as disclosed herein.

6. Multimodal Diagnostic Therapeutic Systems

In one aspect, the compositions of the invention can be a multimodal diagnostic therapeutic system comprising at least one of the solid lipid nanoparticles of the invention. Multimodal diagnostic therapeutic systems can include two or more of the surface active agents of the invention or payloads in at least one solid lipid nanoparticle. That is, more than one function can be achieved by the solid lipid nanoparticle in that it has been adapted to comprise two or more of the biologically active agents, pharmaceutically active agents, magnetically active agents, polyethers, or imaging agents and can also employ the methods of the invention, for example, thermoresponsive payload delivery an/or magnetically-driven targeting.

In one example, a solid lipid nanoparticle can be prepared by the methods of the invention to comprise a biologically active agent, for example a targeting protein; a pharmaceutically active agent, for example a chemotherapeutic; a magnetically active agent, for example magnetite; and an imaging agent, for example a quantum dot. In such an aspect, the solid lipid nanoparticle can achieve the disclosed functions of each component. That is, in such an aspect, the solid lipid nanoparticle can be used to target specific cells, organelles, or tumors; the solid lipid nanoparticle can be used to deliver the chemotherapeutic; the solid lipid nanoparticle can be used in magnetic-driven targeting to a location in a subject; and the solid lipid nanoparticle can be used as an imaging agent.

In one aspect, a single nanoparticle can have the two or more disclosed functions and therefore comprises the multimodal diagnostic therapeutic system. Alternatively, in a further aspect, a mixture of nanoparticles can be prepared, wherein each nanoparticle can have a single disclosed function and the mixture therefore comprises the multimodal diagnostic therapeutic system.

In a further aspect, the compositions of the invention can be a multimodal diagnostic therapeutic system comprising a liposome comprising at least one solid lipid nanoparticle encapsulated within the liposome. In an even further aspect, the compositions of the invention can be a multimodal diagnostic therapeutic system further comprising a delivery package, such as a biologically active agent, a pharmaceutically active agent, a magnetically active agent, imaging agent, or a mixture thereof encapsulated within the liposome. In a still further aspect, the compositions of the invention can be a multimodal diagnostic therapeutic system comprising a microsphere comprising at least one solid lipid nanoparticle encapsulated within the microsphere. In a still further aspect, the compositions of the invention can be a multimodal diagnostic therapeutic system further comprising a delivery package, such as a biologically active agent, a pharmaceutically active agent, a magnetically active agent, imaging agent, or a mixture thereof encapsulated within the microsphere.

Typically, the development and implementation of multi-modal vesicles, for example solid lipid nanoparticles, is highly desired in medicine and biology. For instance, magnetooptical probes consisting of cyanine dye functionalized antibody conjugated to iron oxide nanoparticles was used to detect endothelial surface markers using in vivo confocal microscopy as well as MRI (See Tsourkas, A., V. R. Shinde-Patil, K. A. Kelly, P. Patel, A. Wolley, J. R. Allport, and R. Weissleder, 2005, In vivo imaging of activated endothelium using an anti-VCAM-1 magnetooptical probe, Bioconjug Chem 16(3):576-581). As iron oxide nanoparticles as well as quantum dots can be encapsulated in separate nanoparticle applications, the two can be coencapsulated by introducing CLIO-NP (cross-linked iron oxide-nanoparticle: CLIO-NP is paramagnetic and can also serve as a T2 contrast agent for MRI imaging) in the aqueous phase while quantum dots are introduced in the organic phase prior to phase inversion. This tool represents a major advancement in the field of diagnostic imaging due to the fact that multiple quantum dot emission wavelengths can be excited by only one source, for multi-spectral tracking of cells or proteins in vivo.

Methods of coencapsulating both a magnetically active agent, for example iron oxide nanoparticles, as well as an imaging agent, for example quantum dot nanocrystals, can be based on the method of the invention used to encapsulate each independently within neutral lipids, for example, SOFTISAN®100, SOFTISAN® 142, or SOFTISAN® 154. In brief, water-soluble PEGylated paramagnetic nanoparticles can be introduced into the aqueous phase consisting of PSS, while quantum dots can be placed in the organic phase with NMP/Acetone defined ratios.

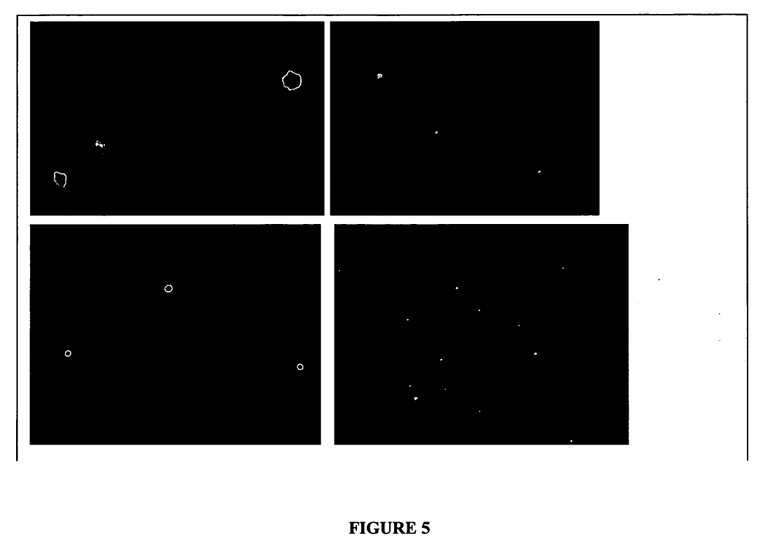

In addition to the development of magnetooptical probes, it can be equally desirable to coencapsulate quantum dots of varying emission wavelengths. In optical barcoding assays, in which each anylate is spectrally coded to distinguish it from its medium (e.g., different cytokines from serum), the ability to have multicolored coding vesicles in addition to single-colored vesicles would increase the maximum number of anylates in the assay. Thus, instead of specimens A, B, and C, one can also prepare AB, AC, and BC as well. Lipid capsules which entrap multiple quantum dots (˜50 QD for a 125 nm lipid nanoparticle as determined by TEM) result in a fluorescent probe with several-fold higher intensities than a single quantum dot (FIG. 5). For this reason, weakly-expressed cell surface antigens not easily detected in vivo can be detectable using this strategy. Additionally, in vitro diagnostic assays can be enabled by this technology. For instance, cell sorting devices which conventionally trigger the counting of an event based on forward and side light scatter properties could now be configured to be triggered upon fluorescence intensities in a certain channel (e.g., a green event, a red event, etc.). This has been achieved using lipid-encapsulated quantum dots detected by a BD FACSAria cell sorter/flow cytometer device.

Functionalized polymers, for example poly(styrene sulfonate) (PSS) and/or poly-L-lysine (PLL), can be successfully attached to the surface of the solid lipid nanoparticles of the invention. The ability to surface engineer PLL in particular can have broad consequences on the ability of this system to bear multiple surface functionalities.

In one aspect, the circulation half-life of a specifically-targeted lipid-antibody probe can be enhanced by the surface engineering of polyethylene-glycol (PEG), a polymer known to reduce immune recognition of nanoparticles and reduce protein adhesion via steric hindrance (See Ballou, B., B. C. Lagerholm, L. A. Ernst, M. P. Bruchez, and A. S. Waggoner, 2004, Noninvasive imaging of quantum dots in mice, Bioconjug Chem 15(1):79-86). A functional PEG reagent known as MALS-PEG-NHS can be conjugated to an antibody via the NHS-ester end of the substance. The resulting antibody-PEG-MALS conjugate can then be attached to a solid lipid nanoparticle probe surface, which bears multiple available amines due to PLL. Such a probe can provide for a method of (a) encapsulation of a drug, nanocrystal-based fluorescent probe (e.g., a quantum dot), or magnetically active agent (e.g., an iron oxide nanoparticle) for contrast enhancement or magnetic control, (b) surface functionalization of a specific protein for the purpose of specific in vivo targeting, and (c) enhanced protection from reticuloendothelial system (See Wang, Y. X. J., S. M. Hussain, and G. P. Krestin, 2001, Superparamagnetic iron oxide contrast agents: physicochemical characteristics and applications in MR imaging, Eur Radiol 11(11):2319-2331) uptake and clearance of the solid lipid nanoparticle probe, thus increasing drug efficacy at the site of interest.

In a further aspect, the solid lipid nanoparticles of the invention can be nanoscale vehicles and can be surface functionalized with one of the well-known cell-penetrating peptides (CPP). CPP have been demonstrated to enter the cytoplasm through an energy-independent mechanism, and include but are not limited to Transportan, Penetratin, and Chariot (See Bolton, S. J., D. N. C. Jones, J. G. Darker, D. S. Eggleston, A. J. Hunter, and F. S. Walsh, 2000, Cellular uptake and spread of the cell-permeable peptide penetratin in adult rat brain, Eur J Neurosci 12(8):2847-2855; Derossi, D., G. Chassaing, and A. Prochiantz, 1998, Trojan peptides: the penetratin system for intracellular delivery, Trends Cell Biol 8(2):84-87; Dom, G., C. Shaw-Jackson, C. Matis, O. Bouffioux, J. J. Picard, A. Prochiantz, M. P. Mingeot-Leclercq, R. Brasseur, and R. Rezsohazy, 2003, Cellular uptake of Antennapedia Penetratin peptides is generally a two-step process in which phase transfer precedes a tryptophan-dependent translocation. Nucleic Acids Res. 31(2):556-561; Langel, U., 2000, In vitro and in vivo applications of transportan as a novel vector, Eur J Neurosci 12:521-521; Letoha, T., S. Gaal, C. Somlai, A. Czajlik, A. Perczel, and B. Penke, 2003, Membrane translocation of penetratin and its derivatives in different cell lines, J. Mol Recognit 16(5):272-279; Lindgren, M., X. Gallet, U. Soomets, M. Hallbrink, E. Brakenhielm, M. Pooga, R. Brasseur, and U. Langel, 2000, Translocation properties of novel cell penetrating transportan and penetratin analogues, Bioconjugate Chem 11(5):619-626; Morris, M. C., J. Depollier, J. Mery, F. Heitz, and G. Divita, 2001, A peptide carrier for the delivery of biologically active proteins into mammalian cells, Nat Biotechnol 19(12):1173-1176; Pooga, M., M. Hallbrink, M. Zorko, and U. Langel, 1998, Cell penetration by transportan, Faseb J 12(1):67-77; Pooga, M., C. Kut, M. Kihlmark, M. Hallbrink, S. Femaeus, R. Raid, T. Land, E. Hallberg, T. Bartfai, and U. Langel, 2001, Cellular translocation of proteins by transportan. Faseb J 15(6); Terrone, D., S. L. W. Sang, L. Roudaia, and J. R. Silvius, 2003, Penetratin and related cell-penetrating cationic peptides can translocate across lipid bilayers in the presence of a transbilayer potential, Biochemistry—Us 42(47):13787-13799; Thoren, P. E. G., D. Persson, M. Karlsson, and B. Norden, 2000, The Antennapedia peptide penetratin translocates across lipid bilayers—the first direct observation, Febs Lett 482(3):265-268).

Without wishing to be bound by theory, it is believed that by adding a peptide system known to facilitate entry into the cytosol to a lipophilic vehicle which by itself is internalized into the cell, the resulting structure can be internalized in a far more efficacious manner. CPP can be initially biotinylated at the N-terminus, using services readily available at peptide synthesis facilities. Next, streptavidin or neutravidin, both biotin-binding proteins, can be covalently coupled to the lipid probe's PLL-coated surface using sulfhlydryl addition to the protein using Traut's reagent (2-iminothiolane) followed by cross-linking using the heterobifunctional reagent sulfo-SMCC (See Hermanson, G. T. 1996. Bioconjugate Techniques. Academic Press, San Diego). The streptavidin/neutravidin-coated lipid nanostructures can then be incubated with a molar excess of biotinylated peptide to complete the conjugation reaction. Further adaptations of this design include the co-functionalization of the lipid surface with an antibody through the cross-linker sulfo-SMCC to facilitate specific targeting on the cell surface (by the antibody), followed by cellular internalization of the conjugate (by the energy-independent peptide and lipid internalization scheme).

In a yet further aspect, extracellular matrix (ECM) degrading enzymes, such as collagenase and MMP-9, can be surface functionalized to the lipid nanoparticle, the interior of which bears a drug, such as Taxol or doxorubicin. Enzymes can be conjugated to the PLL surface of the lipid structures using Traut's reagent sulfhydryl addition in conjunction with sulfo-SMCC cross-linking. The presence of extensive fibrous ECM is well documented in tumors, and serves as a difficult barrier to intratumoral drug penetration. Enzymatic degradation templates on drug carriers have not been previously reported in the literature. Intratumoral injection of lipid capsules which are surface coated with tumor penetrating enzymes which entrap chemotherapeutic agents can serve as a powerful tool for achieving optimal, homogenous tumor drug distribution, through a mechanism which does not necessitate systemic administration which can be associated with adverse side effects and a “dilution” effect of the drug as it passes through the liver and other tissues.

7. Trans-Blood-Brain-Barrier Delivery

Typically, the blood brain barrier (BBB) restricts the transport of large or hydrophilic molecules into the brain. The barrier properties of the BBB are due to the presence of tight junctions. Accordingly, localization of therapeutic and imaging agents into the brain is typically severely restricted.

There are several known mechanisms of crossing the BBB. For example, cells (e.g., Leukocytes) can cross via Cell Migrations. Non-polar solutes, lipid soluble molecules can cross via Passive Diffusion. Lipid Soluble, amphiphilic molecules (including many pharmaceuticals) can cross via Carrier Mediated Efflux. Glucose, amino acids, amines, monocarboxylates, nucleosides, small peptides can cross via Carrier-mediated influx. Transferrin and insulin can cross via Receptor Mediated Transcytosis. Histone, Avidin, and cationized albumin can cross via Adsorptive-mediated transcytosis. Polar solutes can cross via Tight Junction Modulation.

The solid lipid nanoparticles of the invention, by virtue of surface functionalization, neutral lipid character, and nanoscale particle size, can effectively transport a delivery package, such as biologically active agents, pharmaceutically active agents, magnetically active agents, and/or imaging agents across the blood-brain barrier and into the brain tissue.

Lipids and pharmaceutically active agents for preparing the solid lipid nanoparticles of the invention compositions can be dissolved in a binary solvent system comprising, for example, dimethylformamide and acetone. An aqueous solution comprising functionalized polymer, for example poly (acrylic acid), can then be added to the binary solvent solution. A system comprising a solid lipid nanoparticle, a surface functional layer a surrounding the solid lipid nanoparticle, and a pharmaceutically active agent is then formed. The solvents can be removed, thereby yielding a system for delivery of a pharmaceutically active agent across the blood brain barrier.

In one aspect, the methods of the invention can be a method of delivering at least one biologically active agent, pharmaceutically active agent, magnetically active agent, and/or imaging agent across the blood-brain barrier comprising the step of administering an effective amount of the solid lipid nanoparticles of the invention to a subject, whereby the at least one biologically active agent, pharmaceutically active agent, magnetically active agent, or imaging agent is delivered across the blood brain barrier.

8. Trans-Lipid-Bilayer Delivery

Additionally, the solid lipid nanoparticles of the invention, by virtue of surface functionalization, neutral lipid character, and nanoscale particle size, can effectively transport a delivery package, such as biologically active agents, pharmaceutically active agents, magnetically active agents, and/or imaging agents across the lipid-bilayer and into cells.

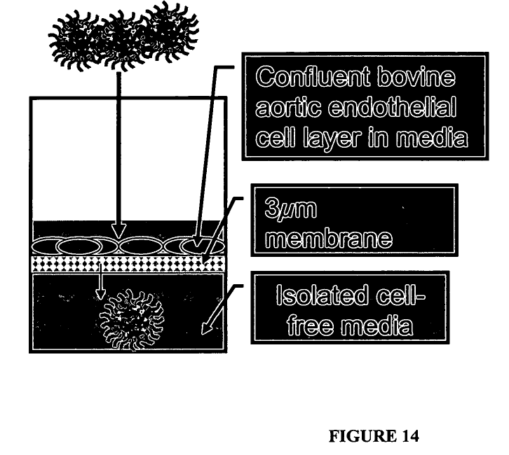

Typically, transport studies can be conducted by (1) SLN solution added at the top of a layer of confluent cells, (2) SLN is then transported across the cell layer, and (3) analysis of the resulting surface modified SLN is performed by microscopy and spectrophotometric analysis. FIG. 14 shows a schematic diagram of such example transport studies for the present method.

In one example, the solid lipid nanoparticles can be functionalized with one or more targeting proteins, such as cell-penetrating peptides (CPP) or proteins including the Nuclear Localization Sequence, as disclosed herein and known to those of skill in the art.

Targeting proteins allow the SLN to penetrate into the nucleus for the purposes of fluorescence in situ hybridization detection (FISH), detection of mRNAs, or staining of nuclear skeleton (lamin A, B, and C, chromatin are examples). See Kalderon D., Richardson W. D., Markham A. F., Smith A. E., Sequence requirements for nuclear location of simian virus 40 large-T antigen, Nature, 311(5981):33-38; Kalderon D., Roberts B. L., Richardson W. D., Smith A. E., A short amino acid sequence able to specify nuclear location, Cell, December 1984; 39(3 Pt 2):499-509.

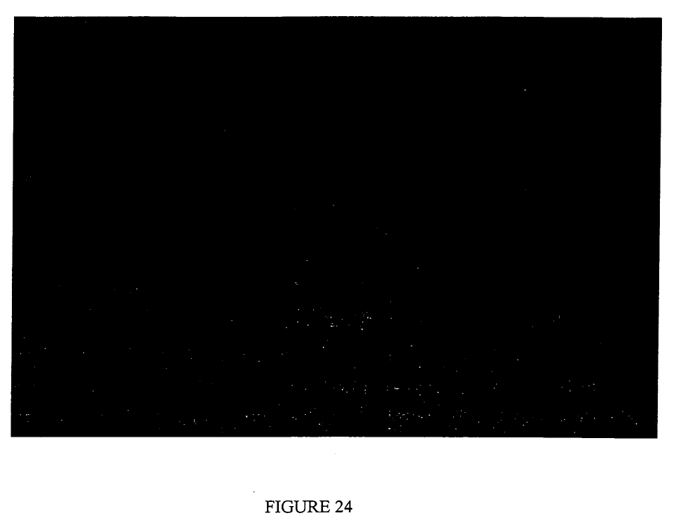

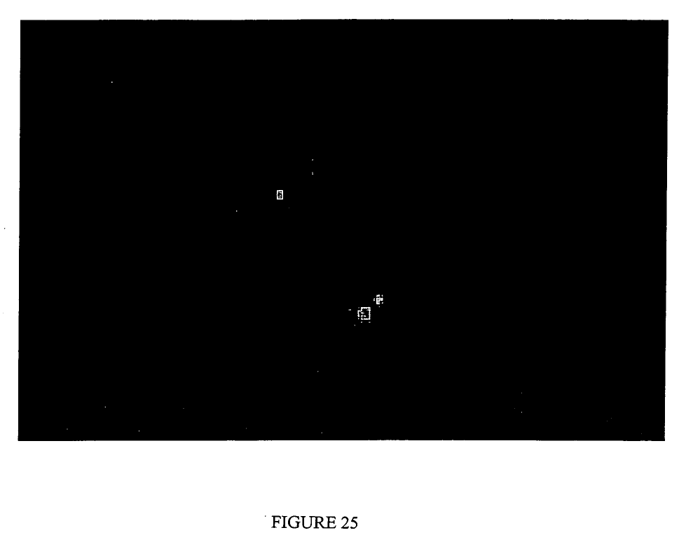

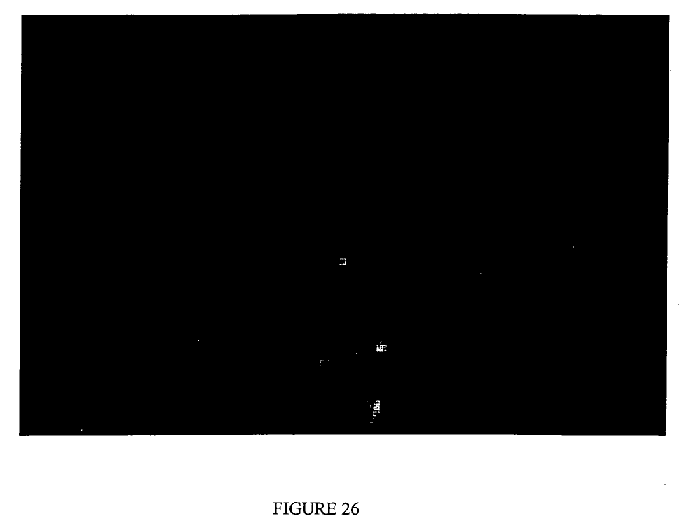

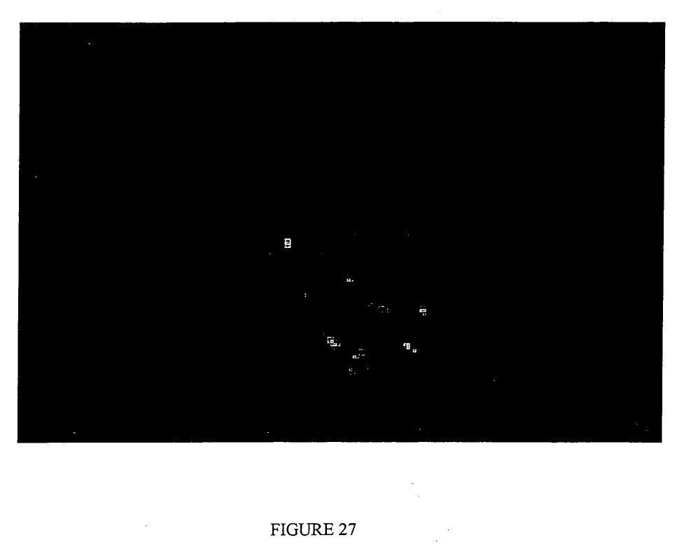

The use of targeting proteins, for example cell-penetrating peptides (CPP), with the solid lipid nanoparticles of the invention enables delivery to the interior of a cell. For example, SLN-QD can be delivered. FIGS. 24-27 show T lymphocytes internalized with lipid-coated QD of the invention attached to CPPs.

In one aspect, the methods of the invention can be a method of delivering at least one biologically active agent, pharmaceutically active agent, magnetically active agent, or imaging agent across a cellular lipid bilayer and into a cell comprising the step of introducing the solid lipid nanoparticles of the invention proximate to the exterior of the cell, whereby the at least one biologically active or pharmaceutically active agents is delivered across the cellular lipid bilayer and into the cell.

9. Subcellular Organelle Targeting

Additionally, the solid lipid nanoparticles of the invention can be used to target specific structures within the interior of a cell. In one aspect, the solid lipid nanoparticles can be functionalized with a targeting protein, such as cell-penetrating peptides (CPP) or proteins including the Nuclear Localization Sequence, and an antibody specific for a subcellular organelle. Antibodies for targeting organelles, also referred to as organelle probes, are well known to those of skill in the art and can be obtained commercially from Invitrogen, for example, as anti-golgin-97 (human), mouse IgG1, monoclonal CDF4 (anti-Golgi), BODIPY® FL C5-ganglioside GM1 complexed to BSA, brefeldin A, N-((4-(4,4-difluoro-5-(2-thienyl)-4-bora-3a,4a-diaza-s-indacene-3-yl)phenoxy)acetyl)sphingosine (BODIPY® TR ceramide), ER-Tracker™ Green (BODIPY® FL glibenclamide), SelectFX® Alexa Fluor® 488 Endoplasmic Reticulum Labeling Kit, anti-OxPhos Complex 117 kDa subunit, mouse IgG2b, monoclonal 21C11, anti-OxPhos Complex IV subunit I, mouse IgG2a, monoclonal 1D6, Alexa Fluor® 594 conjugate (anti-cytochrome oxidase subunit I, Alexa Fluor® 594 conjugate), anti-pyruvate dehydrogenase E1α subunit (human mitochondrial), mouse IgG1, monoclonal 9H9 (anti-PDH E1α subunit), or the like.

In this aspect, the solid lipid nanoparticle can penetrate the cell lipid bilayer and further target a specific subcellular organelle for delivery of a payload. In further aspects, by selecting appropriate antibody clones specific for an organelles, also referred to as signal proteins, or poly(styrene-4-sodium sulfonate), subcellular structures, for example a nucleus, Golgi, endoplasmic reticulum, or mitochondria, can be targeted for delivery of the solid lipid nanoparticles of the invention.

In a further aspect, the methods of the invention can be a method of delivering at least one pharmaceutically active agent, magnetically active agent, or imaging agent to a subcellular organelle comprising the step of introducing the solid lipid nanoparticle of the invention proximate to the exterior of the cell, wherein the solid lipid nanoparticle further comprises at least one pharmaceutically active agent, magnetically active agent, imaging agent, or mixture thereof, and wherein the biologically active agent comprises a signal protein or a targeting protein specific for the organelle, whereby the at least one pharmaceutically active agent, magnetically active agent, imaging agent, or mixture thereof is delivered to the subcellular organelle.

10. Therapeutic Diagnostic Systems

Typically, the solid lipid nanoparticles of the invention are provided as a stable aqueous suspension. Given the hydrophobic character of the solid lipid nanoparticles, hydrophobic interactions with a hydrophobic surface can drive the solid lipid nanoparticles out of the aqueous medium and into contact with the hydrophobic surface, thereby providing a film of the solid lipid nanoparticles of the invention on the surface. Such a film and surface can comprise a therapeutic diagnostic system comprising a hydrophobic polymer substrate and the solid lipid nanoparticles of the invention adsorbed on the surface of the substrate.

11. Cosmetic Formulations

In addition to biological applications, the disclosed nanoparticle compositions can be used in connection with cosmetic applications. By virtue of surface functionalization, neutral lipid character, and nanoscale particle size, the compositions of the invention can effectively transport a delivery package, such as biologically active agents, pharmaceutically active agents, magnetically active agents, and/or imaging agents across the dermis and into the subdermal tissue. Active ingredients having cosmetic activity are well-known in the art and any such ingredient can be used in connection with the compositions of the invention and methods.

In one aspect, the compositions of the invention can comprise a cosmetic formulation comprising the solid lipid nanoparticles of the invention and an active ingredient having cosmetic activity, pharmaceutical activity, or both. Active ingredients having cosmetic activity typically provide moisturizing, depigmenting and/or antibacterial activity. Examples of active ingredient having cosmetic activity include antioxidants, bioprecursors of these antioxidants, for example Δ-tocopherylglucopyranoside, surfactants, fatty substances, moisturizers, preserving agents, fragrances, gelling agents, chelating agents, pigments, for example titanium oxide, screening agents, anti-inflammatory agents, agents to prevent cellular proliferation, anti-UV agents, anti-viral agents, anti-microbial agents, and free vitamins, for example ascorbic acid or α-tocopherol. An active ingredient having pharmaceutical activity is a pharmaceutically active agent.

In a further aspect, the compositions of the invention can comprise a method for the treatment of the upper layers of the epidermis comprising the step of topically administering to a subject an amount effective to treat the upper layers of the epidermis of a composition comprising the disclosed cosmetic formulations.

12. Ink Formulations

Additionally, the compositions of the invention and methods can be used in connection with ink formulations. That is, a dye, a pigment, or a colorant can be encapsulated within the solid lipid nanoparticles of the invention, thereby providing a stable and uniform suspension of the dye within an aqueous system. In one aspect, nanoparticles encapsulating a dye, pigment, or colorant can be included with the disclosed magnetic-driven targeting systems, thereby providing dye systems that can be directed by an externally applied magnetic field. In a further aspect, nanoparticles encapsulating a dye, pigment, or colorant can be included in the disclosed trans-lipid-bilayer delivery systems or subcellular organelle targeting systems, thereby providing dye systems that can deliver staining materials into cells or deliver staining materials to subcellular structures. Further, the lipid character of the resultant composition can provide additional properties to the ink composition, for example, modified melting temperature or superior gloss.

In one aspect, the compositions of the invention can comprise the solid lipid nanoparticles of the invention further comprising a dye, a pigment, or a colorant or stabilized ink compositions comprising the solid lipid nanoparticles of the invention.

E. Experimental

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Preparation of Solid Lipid Nanoparticles

In one aspect, the preparation of the solid lipid nanoparticles of the invention involves preparation and combination of an organic phase and an aqueous phase.

Typically, the organic phase comprises the hydrophobic components, for example, a neutral lipid and any hydrophobic surface active agents and payloads. The components can be selected in any combination deemed to have the appropriate biological interactions, surface chemistries, surface functionalization, surface engineering, size (as measured by hydrodynamic radius, as acquired by Delsa 440SX or Malvern Nanosizer S dynamic light scattering), and/or hydrophilic-lipophilic balance. In a further aspect, the lipid is pre-weighed and dissolved in the organic solvent, for example toluene, such that its final composition in the organic phase is approximately 1% (w/v).

In one aspect, the organic solvent system comprises a binary solvent system, with solvents selected from, for example, those such as toluene, acetone, dimethylformamide, and N-methyl pyrrolidone, to have the desired solvent polarity parameter. A typical solvent system can have a ratio of solvent of about 500 μl toluene to about 200 μl acetone to about 300 μL NMP. The toluene can have a lipid pre-mixed within it, as well as a payload component, for example, a small amount of about 1 μM CdSe/ZnS core-shell quantum dots, which are originally supplied with toluene. A common amount used is about 10 μL, which is counted towards the 500 μL total of toluene; however, it is understood that the amount of solvent can vary to accommodate the desired reaction scale. The organic phase can be prepared usually in an inert environment, such as a glove bag, under a fume hood, and sealed with a septum until use.

In one aspect, the aqueous phase comprises water and any hydrophilic species to be encapsulated or surface functionalized such as therapeutic proteins, magnetic resonance contrast agents (such as Gd-DTPA or iron oxide NP), or water-soluble quantum dots. In a further aspect, also included within this phase are the surface stabilizing species that can be used as a template for layer-by-layer assembly or surface engineering; these can include poly(styrene-4-sodium sulfonate), poly(acrylic acid), poly-L-lysine, etc., as described herein. The polymer composition normally is about 0.1% to about 1% of the total aqueous reaction volume, which is typically about 1 mL. It is understood that the volume of the aqueous phase can vary to accommodate the desired reaction scale.

In a further aspect, magnetic iron oxide NPs can be dispersed within the aqueous solution by gentle vortexing, at a concentration of from about 10 to about 100 μg/mL. This phase can be stirred in a beaker and then transferred to, for example, a BD Vacutainer siliconized/no additive glass tube which has been placed under vacuum to contain no air. Furthermore, prior to introduction in the Vacutainer, the aqueous phase can be degassed to remove dissolved oxygen, to avoid bubbles which may interfere with column purification techniques as well as encapsulation and degradation (via potential oxidation), although degassing is not necessary for proper encapsulation and storage, since the lipids used are effectively resistant to oxidation.

In one aspect, the organic phase can then be drawn into a syringe and injected slowly (1 mL volume) into the BD Vacutainer as it is gently vortexing. The phase is drawn in l0 by vacuum to achieve a relatively controlled flow rate. Vortexing can then be performed for about 25 seconds, for example, after which the lipid suspension is dialyzed first in 20,000 MWCO regenerated cellulose columns for 1 hr in 4 L of water, slowly stirred (<100 rpm) to remove organic solvents from the mixture. Following this process, the solution can then be transferred to a 100K or 300K MWCO cellulose ester float-a-lyzer to remove excess polymers, proteins, and lipids. The SLN can then be filtered for a specific size range using a 0.22 μm or 0.45 μm, 1 μm, etc., syringe filter, and/or analyzed for size and surface charge via dynamic light scattering.

2. Transport

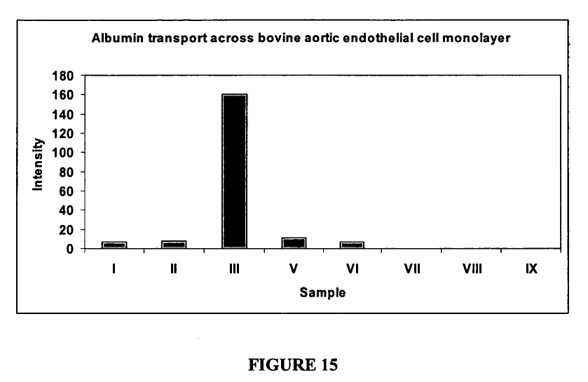

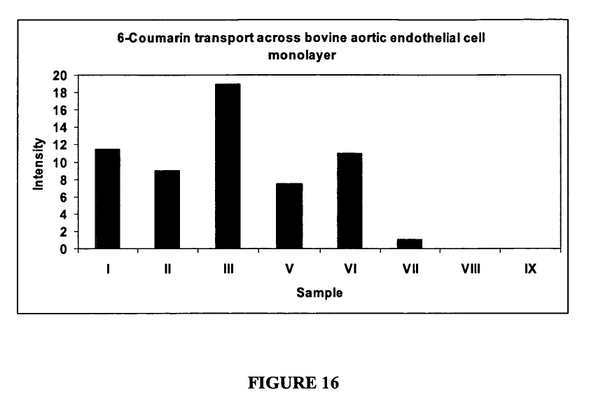

As a specific example, using SLN with the appropriate surface chemistry, a ten-fold increase in albumin transport can be achieved over polyethylene glycol (PEG)- or poly (vinyl alcohol) (PVA)-coated SLN. This result is shown in the graph of FIG. 15. Transport of coumarin, a lipophilic molecule, follows a similar trend, as shown in FIG. 16.

3. Imaging

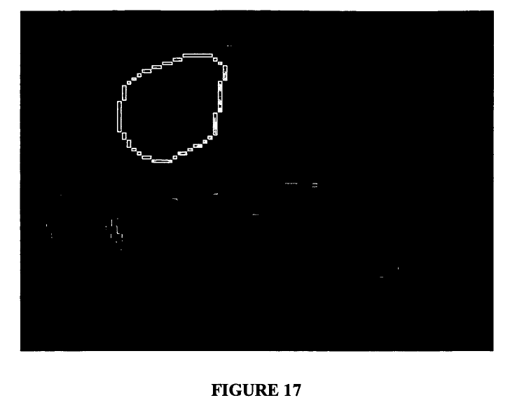

MRI imaging studies using functionalized SLN were performed by encapsulating Gadolinium DTPA in SLN bearing various surface functionalities. Mouse brain was then imaged in the axial position. The images were gradient-echo, TE/TR=2.5/250 ms, 45 degree flip angle, 4 averaged excitations per image, 128×128 over a 20 mm field of view. Each image was acquired over 128 seconds. The time course accounts for the extra delay between runs of 12 images each. An example resultant MRI image is shown in FIG. 17.

4. Encapsulation of a Magnetically Active Agent

250 nm dextran cross-linked iron oxide nanoparticles (CLIO, Micromod GmbH) were encapsulated in a triglyceride matrix consisting of SOFTISAN® 100 (Sasol GmbH) using a phase inversion process described previously. Nanoparticles were vortexed for 30 seconds in phosphate buffered saline (PBS) at a pH of 7.4, and introduced at a 100 ug/mL concentration into an aqueous phase consisting of 1% (w/v) fluorescein isothiocyanate-labeled bovine serum albumin (FITC-BSA) as a fluorescent indicator and 1% (w/v) poly(styrene-4-sodiumsulfonate) (PSS), a negatively-charged polymeric surface functionality which confers enhanced water solubility upon lipid nanoparticles. An organic phase consisting of the SOFTISAN®100 lipid at 1% (w/v) in a mixture of anhydrous n-methyl-pyrrolidone and acetone was prepared and introduced into the aqueous phase concurrent with gentle vortexing to encourage phase inversion. The resulting dispersion of lipid nanoparticles was dialyzed against ultrapure distilled water in 20,000 molecular weight cutoff (MWCO) regenerated cellulose dialysis columns to remove organic solvents from the lipid dispersion. The dialyzed solution was then dialyzed against ultrapure distilled water in 100,000 MWCO cellulose ester dialysis columns to remove unencapsulated FITC-BSA. The resulting lipid dispersion was filtered using macroporous (70 um) filter paper to remove lipid aggregates.

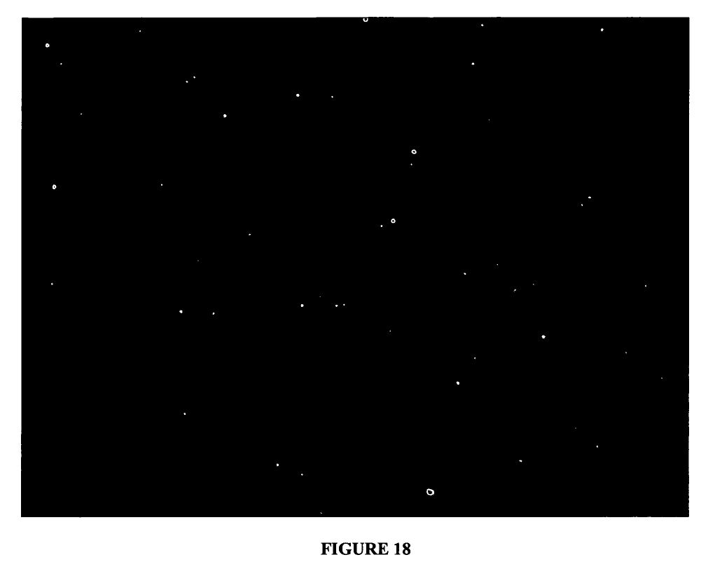

The paramagnetic nanoparticles produced above were successfully encapsulated, as confirmed by fluorescence microscopy. Trypan blue at a 0.5M concentration was incubated for 10 minutes with FITC-BSA/iron oxide encapsulated nanoparticles to quench unencapsulated FITC-BSA fluorescence. Fluorescence microscopy of lipid encapsulations of iron oxide nanoparticles and FITC-BSA indicated a punctate pattern of unquenched FITC-BSA fluorescence (see FIG. 18), which indicates that lipid entrapment of FITC-BSA has occurred, which isolates the protein from a quenching environment. A control solution involving the same components as the above solution without the lipid indicated negligible fluorescence, which indicates that either FITC-BSA was removed completely by dialysis, and/or fluorescence from the protein was quenched by trypan blue.

Upon application of a magnetic field using a static magnet (1.5 T), FITC-BSA nanoparticles shown in FIG. 18 were observed to move in response to magnet polar orientation. Fluorescent nanoparticles were highly sensitive to rapid changes in the external field while control nanoparticles consisting of FITC-BSA mixed with CLIO-NP in distilled water without lipid did not move in response to changes in magnetic fields. These data indicate that FITC-BSA was co-encapsulated with CLIO-NP, and that magnetization properties of CLIO-NP are not affected by encapsulation.

5. Aggregation Studies

Aggregation studies of the lipid-encapsulated paramagnetic nanoparticles of the present invention indicate that upon application of an external magnetic field, lipid-encapsulated species do not readily aggregate compared to unencapsulated CLIO-NP. Suspensions of the present lipid-encapsulated paramagnetic nanoparticles and unencapsulated CLIO-NP were prepared in 20 mL scintillation vials and were placed above a static magnet (1.5T) for 10 minutes. After removal of the magnet, CLIO-NP formed a thick black film along the bottom of the vial. Lipid NP also formed a milky black film on the bottom. Gentle tapping of the vial removed the lipid-NP film, which resuspended readily in solution, but not the CLIO-NP film. These observations demonstrate that lipid-encapsulated paramagnetic nanoparticles provide a mechanism for reduced irreversible aggregation that is associated with currently-used nanoparticle regimens. Without wishing to be bound by theory, it is believed that the repulsion between lipid vesicles is due to the highly negatively-charged coating conferred by the surface functionalization of poly(styrene-4-sodium-sulfonate).

6. Preparation and Use of SLN-QD

a. Preparation

Cadmium-selenide zinc-sulphide core-shell quantum dot semiconducting nanocrystals were purchased from Evident Technologies (Troy, N.Y.) in toluene. In one example, an organic phase consisting of a fixed ratio of n-methyl pyrrolidone and acetone, anhydrous, 1% SOFTISAN® 100 or 142, and 1 μM CdSe/ZnS quantum dots was prepared and stored over 3A molecular sieves until use, and introduced into a vortexing aqueous phase consisting of 1% PSS. The mixture was vortexed for about 10 seconds to encourage mixing of phases. The suspension was then filtered through macroporous 70 μm filter paper to remove lipid aggregates, then dialyzed against ultrapure distilled water by 20,000 MWCO regenerated cellulose dialysis columns to remove organic solvents, followed by dialysis against ultrapure distilled water with 100,000 MWCO cellulose ester dialysis column to remove excess polymer.

In a further example, cadmium selenide-zinc sulfide (CdSe—ZnS) core-shell nanocrystals emitting at 580 nm (Evident Technologies) were immersed in a binary solvent system consisting of anhydrous solutions of 1-methyl-2-pyrrolidone (NMP) and acetone (Sigma). This solution was infused with an aqueous phase containing poly(styrene-4-sulfonate) (PSS) to form a stable microemulsion. Phase inversion resulted in the instantaneous packing of the lipid moieties into colloids, within which quantum dots were entrapped. High molecular weight dialysis using Spectrapor Float-a-Lyzers was performed to remove unconjugated species. Molar ratios of the two solvents were varied to explore effects on nanoparticle size.

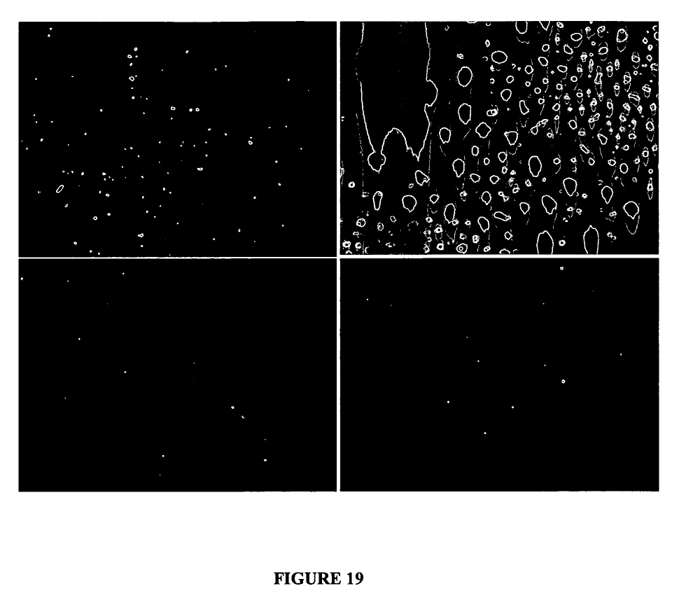

Fluorescence microscopy indicates that quantum dots were successfully encapsulated by the disclosed process. In previous methods, exposure to an aqueous environment results in aggregation of the hydrophobic quantum dots, resulting in their disintegration. Thus, when engineering quantum dot vehicles, it can be necessary to protect them from the aqueous environment by a water solubilization scheme. As shown in FIG. 19, when quantum dots entrapped by the lipid encapsulation process are subjected to an injection of lactated Ringers' buffer, no aggregation associated with uncoated quantum dots is observed. Unencapsulated quantum dots in toluene exposed to Ringers', on the other hand, are observed to rapidly aggregate and disintegrate. This data indicates that the lipid enclosure effectively surrounds the quantum dots, and serves as a protective barrier from the degradative aqueous environment. Quantum dots encapsulated by the disclosed processes remain fluorescent and intense for up to six months or more with storage at room temperature in a cool storage area protected from light. Storage under inert gas or with antioxidants is not necessary.

Further confirmation of successful quantum dot encapsulation was provided by spectrofluorimetry (Nanodrop, Inc.), which showed peak emission of dialyzed and filtered lipid-encapsulated quantum dots at the same peak emission of unencapsulated quantum dots in toluene. This provides evidence that quantum dots were successfully encapsulated by the disclosed process, and also that lipid encapsulation does not alter the highly desirable optical properties of the nanocrystals. The encapsulated quantum dots did not degrade with storage, and were observed to retain fluorescence intensity even after six months in storage at room temperature.

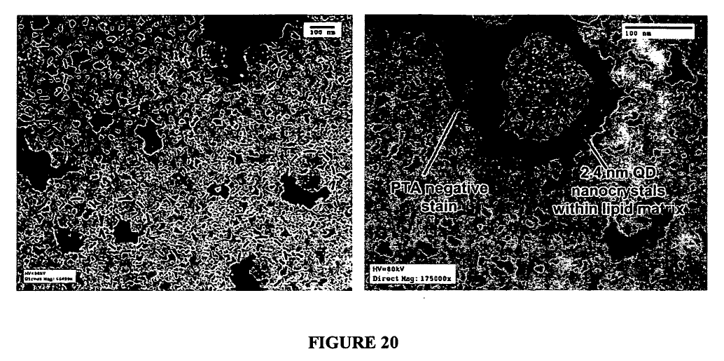

Transmission electron microscopy of SLN-QD is also indicative of successful quantum dot encapsulation by the lipid. Phosphotungstic acid was used to label the lipid coating for observation of vesicles by negative relief (see FIG. 20). Quantum dots are dense, 2 nm rods which are dark due to their electron density, and are surrounded by a white cloud of lipid. A lower magnification view of the sample indicates that most nanoparticles produced by this process are of similar diameter, and quantum dots are not aggregated within the lipid entrapment, but rather are dispersed in a “honeycomb” pattern, which is useful in the preservation of quantum dot optical properties observed during spectrofluorimetry experiments on the same sample.

Particle diameter analysis (Beckman-Coulter Delsa 440SX Zetasizer) indicates that small changes in solvent polarity due to the adjustment of n-methyl-pyrrolidone/acetone ratios in the organic phase result in proportional changes in nanoparticle diameter (see FIG. 2). It is hypothesized that alterations in solvent ratios control the diffusivity of water into the organic phase, which thus controls the size of the packed lipid structures as hydrophobic species cluster together. Fluorescence microscopy of lipid-encapsulated quantum dots prepared with different organic solvent ratios were noticeably different in size by qualitative analysis (see FIG. 5). With the example processes, nanoparticle diameters from about 10 nm to about 1000 nm, for example from about 50 nm to as high as about 700 nm, have been produced.

b. Internalization of SLN-QD in Cells

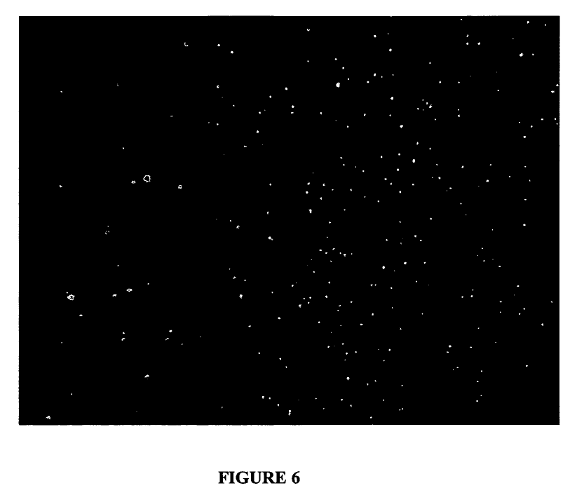

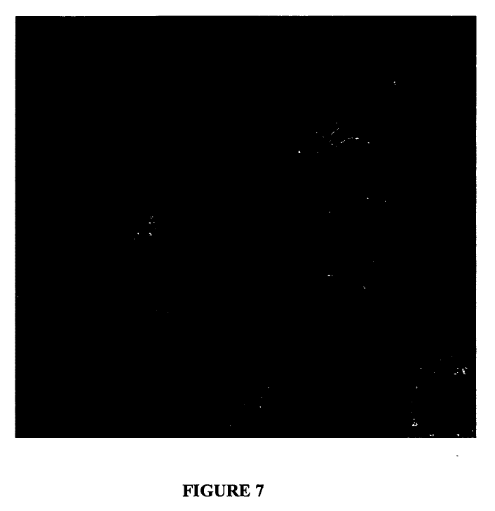

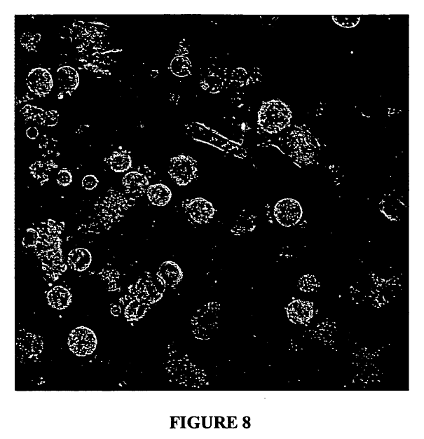

Incubation of PSS-coated lipid-encapsulated quantum dots with bovine aortic endothelial cells (BAEC) for 2 hours at 37° C. followed by thorough rinsing in PBS resulted in internalization of the lipid moieties (FIG. 6). Fluorescence microscopy of live cells (FIG. 7) indicates dense perinuclear staining within the cells. Furthermore, the quantum dots were not removed with trypsinization of BAEC (FIG. 8), which indicates that the quantum dots were not associated with plasma membrane but rather the cytoplasm of the cell following incubation.

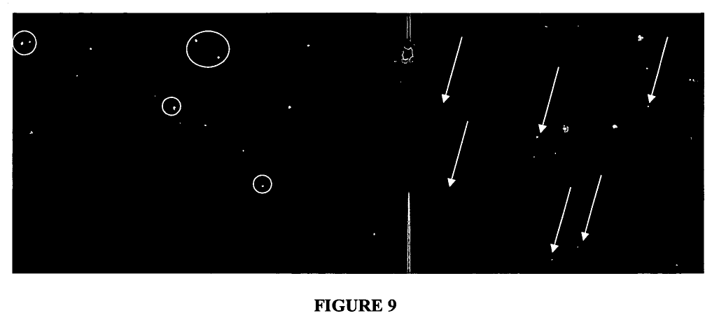

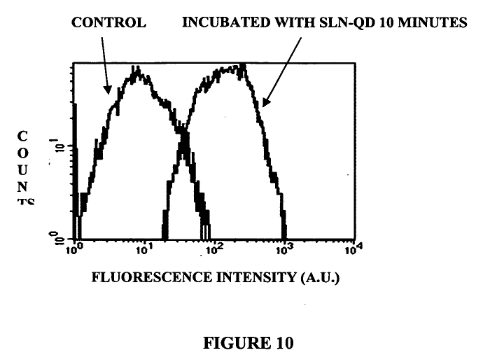

To determine the speed of cellular internalization of lipid-encapsulated quantum dots, live CCD microscopy was performed to visualize BAEC internalization of the vesicles (FIG. 9). After 10 minutes, Brownian motion of the lipid nanoparticles slowed down rapidly and cells became embedded within plasma membranes. Flow cytometry of BAEC incubated for only 10 minutes with our quantum dot structures indicated significant rapid internalization (FIG. 10). Cellular uptake of lipid-entrapped quantum dots is a rapid process.

C. Flow Cytometry

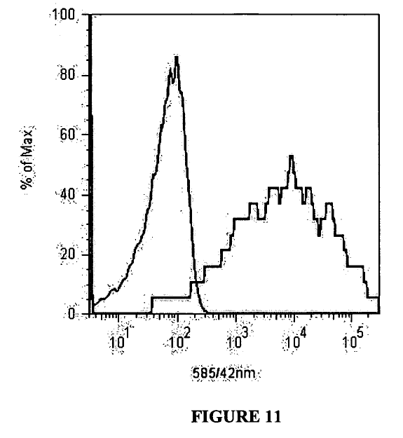

The dependence of cellular internalization on energy (i.e., endocytosis) was investigated. BAEC were incubated with the same sample used in flow cytometry and microscopy at 4° C. Flow cytometry of the sample after 25 minutes was indicative of quantum dot uptake relative to control, unincubated cells (FIG. 11). A control, Qtracker (Quantum Dot Corporation), a peptide-coated quantum dot solution which relies on endocytosis, was shown not to be taken up by cells to at the same temperature, as analyzed by flow cytometry and fluorescence microscopy. Incubation at 4° C. is an accepted method for studying the uptake of peptides (e.g., Chariot) by energy-independent mechanisms (See Morris, M. C., J. Depollier, J. Mery, F. Heitz, and G. Divita, 2001, A peptide carrier for the delivery of biologically active proteins into mammalian cells, Nat Biotechnol 19(12):1173-1176). Thus, the internalization of the solid lipid nanoparticles of the invention is at least in part an energy-independent process.

d. Zeta Potential

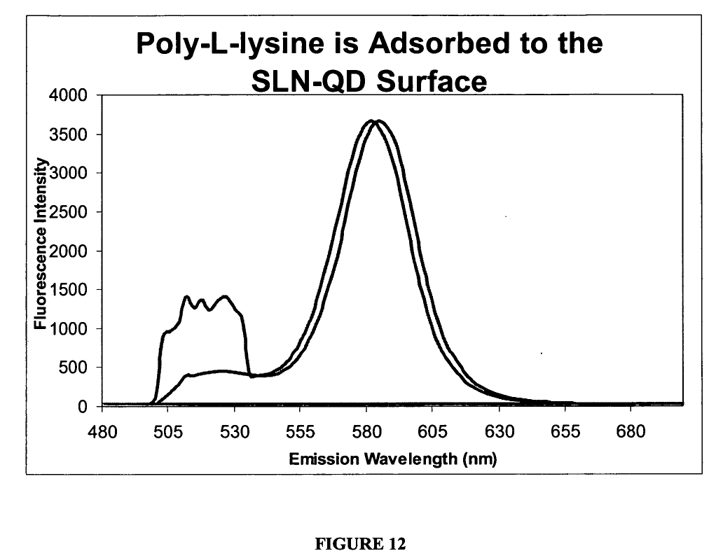

Both iron oxide nanoparticle and quantum dot lipid encapsulations were analyzed for zeta potential values to confirm the presence of PSS on the lipid surface following dialysis and filtration. FIG. 12 shows the zeta potential profile for lipid-encapsulated quantum dots coated with PSS. The profile is indicative of charge neutrality at the pKa of the ionizable group, sulfonic acid, which is present in PSS. As the isoelectric point of the sample corresponds with the pKa of sulfonic acid, it follows that a negatively-charged template can be engineered to the lipid vesicle surface. Upon this negatively-charged polymer surface, it was demonstrated that poly-L-lysine, a highly desirable template for bioconjugation due to the presence of amine groups, could be successfully electrostatically adsorbed. This has been demonstrated by spectrofluorimetry as well as zeta potential analysis of specimens. This system can present multiple polymeric templates, which include negatively charged, positively charged, and mucoadhesive polymers.

e. Surface Engineering of SLN-QD

SLN-QD prepared by the method above contained negatively-charged PSS templates upon which further surface functionalization could be achieved. SLN-QD were immersed in an aqueous solution containing positively charged poly-L-lysine (PLL), which electrostatically adsorbed to the PSS surface to create a positively-charged template. See FIG. 12.

f. Surface Charge and Size Measurements

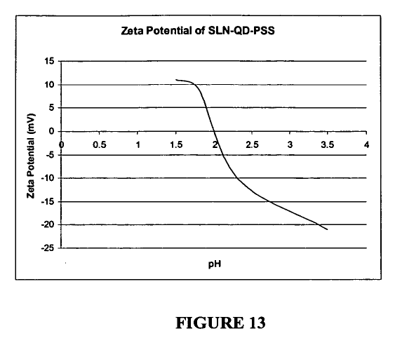

Measurements of SLN-QD surface charge were performed using a Beckman-Coulter Delsa 440SX zetasizer. Samples were measured for zeta potential and nanoparticle diameter. SLN-QD preparation for TEM was performed by plating on Formvar grids with phosphotungstic acid (PTA), a negative lipid stain. Nanoparticle loading density in stained SLN-QD was estimated by counting of quantum dots in each sphere, from TEM images. See FIG. 2 and FIG. 13.

g. Spectral Properties and Stability of SLN-QD

Absorbance measurements of the prepared SLN-QD were performed on a Nanodrop ND-1000 spectrophotometer to detect any changes in quantum dot optical properties. Fluorescence intensity of the solid lipid nanoparticles in the visible spectrum was measured using a Nanodrop fluorimeter. Results were compared to unencapsulated core-shell nanocrystals in toluene.

Unencapsulated and encapsulated nanocrystals were subjected to an influx of lactated Ringers, an aqueous buffer, and were observed by live CCD imaging coupled to an inverted fluorescence microscope, to detect possible aggregation associated with hydrophobic quantum dot interactions with water.

h. Live Cell Labeling Using SLN-QD

Bovine aortic endothelial cells (BAEC) were cultured to confluency on chambered coverslips (Nunc). Cells were incubated with SLN-QD for intervals between 10 minutes and 1 hour. Cultures were rinsed three times with phosphate buffered saline. A fraction of the cells were fixed for observation by confocal microscopy, while another fraction was quantified for SLN-QD fluorescence using a BD FACSCalibur flow cytometer. Using an inverted fluorescence microscope (Nikon TE 2000U) with a color CCD imaging system (Hamamatsu), live cells were incubated with SLN-QD and were recorded for 1 hour with an Exfo metal halide excitation lamp (Exfo), 488 excitation filter (Chroma) and an emission filter tuned to the SLN-QD passband of 580/20 (Omega Optical). Control cells incubated with an equivalent amount of SLN-QD storage buffer without quantum dots were also labeled and fixed for imaging, as well as labeled in vitro for live CCD recording.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A solid lipid nanoparticle comprising:
a. a neutral lipid and
b. a first functionalized polymer,
wherein the solid lipid nanoparticle has an interior, an exterior, and a diameter;
wherein the first functionalized polymer comprises at least one ionic or ionizable moiety;
wherein at least a portion of the first functionalized polymer is at the exterior of the solid lipid nanoparticle; and
wherein the diameter of the solid lipid nanoparticle is from about 10 nm to about 1,000 nm.

2. The solid lipid nanoparticle of claim 1, wherein at least a portion of the first functionalized polymer is embedded in the lipid.

3. The solid lipid nanoparticle of claim 1, wherein at least one ionic or ionizable moiety is at the exterior of the solid lipid nanoparticle.

4. The solid lipid nanoparticle of claim 1, wherein the lipid comprises a monoglyceride, diglyceride, or triglyceride of at least one C₄ to C₂₄ carboxylic acid.

5. The solid lipid nanoparticle of claim 1, wherein the lipid comprises a triglyceride of at least one saturated, even-numbered, unbranched natural fatty acid with a chain length of C₈ to C₁₈.

6. The solid lipid nanoparticle of claim 1, wherein the first functionalized polymer comprises a polymer having at least one ionic or ionizable pendant group.

7. The solid lipid nanoparticle of claim 1, wherein the first functionalized polymer comprises a polymer having at least one ionic or ionizable moiety in the polymer backbone.

8. The solid lipid nanoparticle of claim 1, wherein the first functionalized polymer further comprises a biologically active agent.

9. The solid lipid nanoparticle of claim 1, wherein the first functionalized polymer further comprises a pharmaceutically active agent.

10. The solid lipid nanoparticle of claim 1, further comprising a second functionalized polymer substantially surrounding the first functionalized polymer.

11. The solid lipid nanoparticle of claim 10, wherein the second functionalized polymer further comprises a biologically active agent.

12. The solid lipid nanoparticle of claim 10, wherein the second functionalized polymer further comprises a pharmaceutically active agent.

13. The solid lipid nanoparticle of claim 10, further comprising a third functionalized polymer substantially surrounding the second functionalized polymer.

14. The solid lipid nanoparticle of claim 13, wherein the third functionalized polymer comprises a biologically active agent.

15. The solid lipid nanoparticle of claim 13, wherein the third functionalized polymer comprises a pharmaceutically active agent.

16. The solid lipid nanoparticle of claim 1, further comprising a payload encapsulated within the interior of the particle.

17. The solid lipid nanoparticle of claim 16, wherein the payload comprises a pharmaceutically active agent.

18. The solid lipid nanoparticle of claim 16, wherein the payload comprises a magnetically active agent.

19. The solid lipid nanoparticle of claim 16, wherein the payload comprises an imaging agent.

20. The solid lipid nanoparticle of claim 16, wherein the imaging agent comprises a quantum dot.

21. A solid lipid nanoparticle comprising a neutral lipid and a polyether; wherein the solid lipid nanoparticle has an interior, an exterior, and a diameter; wherein at least a portion of the polyether is at the exterior of the solid lipid nanoparticle; and wherein the diameter of the solid lipid nanoparticle is from about 10 nm to about 1,000 nm.

22. The solid lipid nanoparticle of claim 21, further comprising at least one of a biologically active agent, a pharmaceutically active agent, a magnetically active agent, or an imaging agent, or a mixture thereof.

23. A functionalized quantum dot comprising one or more quantum dots encapsulated within the solid lipid nanoparticle of claim 1.

24. The functionalized quantum dot of claim 23, wherein the first functionalized layer further comprises at least one cysteine-rich protein, at least one metallothionein-rich protein, or a mixture thereof.

25. A tumor targeting therapeutic system comprising:
a. a solid lipid nanoparticle of claim 8, and
b. a pharmaceutically active agent encapsulated within the solid lipid nanoparticle;
wherein the biologically active agent comprises at least one enzyme.

26. A multimodal diagnostic therapeutic system comprising at least one solid lipid nanoparticle of claim 1, wherein the at least one solid lipid nanoparticle is optionally encapsulated within a liposome or a microsphere.

27. The multimodal diagnostic therapeutic system of claim 26, further comprising a biologically active agent, a pharmaceutically active agent, a magnetically active agent, imaging agent, or a mixture thereof encapsulated within the liposome or a microsphere.

28. A thermoresponsive payload delivery system comprising:
a. a first solid lipid nanoparticle of claim 16, wherein the first solid lipid nanoparticle has a first payload and a first melting temperature.

29. The thermoresponsive payload delivery system of claim 28, further comprising:
b. a second solid lipid nanoparticle of claim 16, wherein the second solid lipid nanoparticle has a second payload and a second melting temperature, and wherein the second melting temperature is higher than the first melting temperature.

30. A method of thermoresponsive payload delivery within a subject comprising the steps of:
a. administering an effective amount of the thermoresponsive payload delivery system of claim 28 to a subject; and
b. applying heat to a location within the subject, thereby increasing the temperature of the location above the first melting temperature and melting the solid lipid nanoparticle, whereby the first payload is delivered to the location within the subject.

31. A method of thermoresponsive payload delivery within a subject comprising the steps of:
a. administering an effective amount of the thermoresponsive payload delivery system of claim 29 to a subject;
b. applying a first heat to a first location within the subject, thereby increasing the temperature of the first location above the first melting temperature and melting the first solid lipid nanoparticle, whereby the first payload is delivered to the first location within the subject; and
c. applying a second heat to a second location within the subject, thereby increasing the temperature of the second location above the second melting temperature and melting the second solid lipid nanoparticle, whereby the second payload is delivered to the second location within the subject.

32. A method of providing a solid lipid nanoparticle comprising the steps of:
a. providing an organic phase comprising: (1) a binary solvent system and (2) a neutral lipid;
b. providing an aqueous phase comprising water and at least one first functionalized polymer having at least one ionic or ionizable moiety; and
c. combining the organic phase and the aqueous phase.

33. The method of claim 32, further comprising the step of:
d. admixing with the product of claim 32 a second functionalized polymer having at least one ionic or ionizable moiety that is complementary to the ionic or ionizable moiety of the first functionalized polymer.

34. The product produced by the method of claim 32.

35. The product produced by the method of claim 33.

36. A method of delivering at least one biologically active agent, pharmaceutically active agent, magnetically active agent, or imaging agent across the blood-brain barrier comprising the step of administering to a subject an effective amount of the solid lipid nanoparticle of claim 1, further comprising at least one biologically active agent, pharmaceutically active agent, magnetically active agent, or imaging agent, whereby the at least one biologically active agent, pharmaceutically active agent, magnetically active agent, or imaging agent is delivered across the blood brain barrier.

37. A method of delivering at least one biologically active agent, pharmaceutically active agent, magnetically active agent, or imaging agent to a location within a subject comprising the steps of:
a. administering an effective amount of the solid lipid nanoparticle of claim 18 to a subject,
b. applying a magnetic field to the location, whereby the at least one biologically active agent, pharmaceutically active agent, magnetically active agent, or imaging agent is delivered to the location.

38. A method of delivering at least one biologically active agent, pharmaceutically active agent, magnetically active agent, or imaging agent across a cellular lipid bilayer and into a cell comprising the step of introducing proximate to the exterior of the cell the solid lipid nanoparticle of claim 1, further comprising at least one biologically active agent, pharmaceutically active agent, magnetically active agent, imaging agent, or mixture thereof, whereby the at least one biologically active agent, pharmaceutically active agent, magnetically active agent, imaging agent, or mixture thereof is delivered across the cellular lipid bilayer and into the cell.

39. A method of delivering at least one pharmaceutically active agent, magnetically active agent, or imaging agent to a subcellular organelle comprising the step of introducing the solid lipid nanoparticle of claim 8 proximate to the exterior of the cell, wherein the solid lipid nanoparticle further comprises at least one pharmaceutically active agent, magnetically active agent, imaging agent, or a mixture thereof, and wherein the biologically active agent comprises a signal protein specific for the organelle, whereby the at least one pharmaceutically active agent, magnetically active agent, imaging agent, or mixture thereof, is delivered to the subcellular organelle.

40. A therapeutic diagnostic system comprising:
a. a hydrophobic polymer substrate and
b. a solid lipid nanoparticle of claim 1 adsorbed on the surface of the substrate.

41. A method of providing the therapeutic diagnostic system of claim 40 comprising the step of contacting an aqueous suspension of a solid lipid nanoparticle of claim 1 with a hydrophobic polymer substrate.

42. A method of modulating particle size of a solid lipid nanoparticle comprising the steps of
a. selecting a binary solvent system;
b. dissolving a neutral lipid in the binary solvent system, thereby producing an organic phase;
c. providing an aqueous phase comprising a first functionalized polymer; and
d. combining the organic phase and the aqueous phase, thereby producing a substantially monodisperse solid lipid nanoparticle suspension.

43. The solid lipid nanoparticle of claim 1, further comprising a dye, a pigment, or a colorant.

44. A stabilized ink composition comprising the solid lipid nanoparticle of claim 43.

45. A cosmetic formulation comprising a solid lipid nanoparticle of claim 1 and an active ingredient having cosmetic activity, pharmaceutical activity, or both.

46. A method for the treatment of the upper layers of the epidermis comprising the step of topically administering to a subject an amount effective to treat the upper layers of the epidermis of a composition comprising a cosmetic formulation of claim 45.
